(19) United States
(12) Patent Application Publication
(10) Pub. No.: US 2010/0144055 A1
(43) Pub. Date: Jun. 10, 2010

(54) Assays For Clinical Assessments of Disease-Associated Autoantibodies

(75) Inventors: Thomas F. Holzman, Libertyville, IL (US); Winton G. Gibbons, Winnetka, IL (US)

Correspondence Address: GREGORY T. PLETTA;Nanosphere, Inc., 4088 Commerical Avenue, Northbrook, IL, 60062-1829, US

(73) Assignee: NANOSPHERE, INC., Northbrook, IL (US)

(21) Appl. No.: 12/498,348

(22) Filed: Jul. 6, 2009

Related U.S. Application Data
(63) Continuation of application No. 12/267,543, filed on Nov. 7, 2008, now abandoned.

(51) Int. Cl. G01N 33/53

(52) U.S. Cl. 436/501

(57) ABSTRACT

The disclosure provides methods of detecting autoantibodies (AAs) in biological samples. The methods use capture probes that can bind to an disease-associated antigen/AA complex and a detection probe that can bind to the AAs. The presence, absence, and/or amount of the complex may be measured, wherein the presence of the complex may be diagnostic or prognostic of a disease or medical condition. The disclosure also provides methods of simultaneously detecting AAs and antigens in biological samples. The presence, absence, and/or amount of AAs and antigens may be measured, wherein the amount of antigen present and/or the amount of autoantibody present may be diagnostic or prognostic of a particular disease or medical condition.

### TECHNICAL FIELD

The present technology relates generally to diagnostic and prognostic methods for human disease. In particular, the present disclosure relates to methods for detecting autoantibodies (AA) which are a marker for a human disease or medical condition.

### BACKGROUND

The following description is provided to assist the understanding of the reader. None of the information provided or references cited is admitted to be prior art to the present invention.

Diseased cells are often characterized by the production of disease-associated marker proteins. Freedland et al. Jama 2005; 294:433-439. These consist of aberrant forms of wild-type proteins, which are produced by disease cells as a result of genetic mutations, alternative haplotypes, or altered post-transcriptional or post-translational processing. Soussi T. Oncogene 2007; 26:2145-2156. Alternatively, disease markers can also be proteins that become over-expressed in diseased cells, usually as a result of gene amplification or abnormal transcriptional regulation. In some cases, these two phenomena may occur at the same time leading to an accumulation of modified proteins throughout the development of the disease. For example, modified forms of Ras, p53, c-myc, MUC-1, c-erbβ2 have been found to be associated with a wide variety of cancers. Kargozaran et al. Surgical Oncology Clinics of North America 2008; 17:341. Likewise, variant forms of filaggrin protein are associated with rheumatoid arthritis (RA). Expression of the disease-associated marker proteins may result in production of AAs against these self-antigens. The AAs may be detected in serum and are therefore useful as diagnostic markers for the disease or condition which resulted in the production of the disease-associated marker protein.

### SUMMARY

The technology disclosed herein relates to the detection of AAs for the diagnosis of disease. The present inventors found that it is possible to detect AAs in diagnostic assays in which the disease-associated marker protein is captured, and the AAs associated with the marker protein are detected. These assays do not require knowledge of the specific genetic mutations, altered alleles, altered haplotypes, altered post-transcriptional or post-translational processing, or altered metabolic states prior to conducting the assay. Hence, the assay is referred to as “autoantibody fishing.” These assays have a much higher sensitivity than currently used tests and are therefore able to detect smaller quantities of the AAs. Furthermore, the inventors have developed specific algorithms that will facilitate the ruling in and ruling out of patients with disease by analyzing multiple markers simultaneously. The present technology can be applied to diagnostic and prognostic assays for any other disease or condition where AAs are generated, such as autoimmune disease and cancer.

In one aspect, the disclosure provides a method for detecting a disease-associated AA present in a sample from a subject comprising: (a) contacting the sample with (i) a capture probe, wherein the capture probe comprises a first binding agent capable of specifically binding a disease-associated antigen and (ii) a detection probe comprising a second binding agent capable of specifically binding AAs to the disease-associated antigen; and (b) detecting the presence of a complex formed between the capture probe, the disease-associated antigen, AAs to the disease-associated antigen, and the detection probe, wherein the presence of the complex is indicative of disease-associated AAs in the sample.

In one embodiment, the disease-associated antigen is a polypeptide associated with autoimmune disease, such as RA, systemic lupus erythematosus (SLE), myasthenia gravis, or Grave's disease. For example, in RA, the polypeptide associated with autoimmune disease may be a filaggrin polypeptide or variant thereof, such as a citrullinated filaggrin polypeptide.

In one embodiment, the disease-associated antigen is a polypeptide associated with cancer. For example, the disease-associated antigen may be p53 or a variant thereof and the AAs specifically bind to the p53 protein or variant thereof. In other examples, the disease-associated antigen may be modified forms of Ras, c-myc, MUC-1, c-erbβ2, or PSA.

In other embodiments, the disease-associated antigen is associated with cardiovascular disease. The disease-associated antigen may be a cardiac marker well known in the art, including but not limited to, cardiac troponins, brain naturetic peptide, etc.

In other embodiments, the disease-associated antigen is a polypeptide associated with a neurodegenerative disorder. For example, the disease-associated antigen may be phospho tau; amyloid beta; alpha-synuclein; protease-resistant prions; superoxide; dismutase-1, huntingtin, ataxin, or other antigens known in the art.

In one embodiment, the first binding agent is an antibody, antibody fragment, aptamer, or polypeptide. For example, the first binding agent may be a polyclonal antibody raised against a disease-associated antigen. Alternatively, the first binding agent may be monoclonal antibody raised against a conserved region of the disease-associated antigen. Binding a conserved region of a specific antigen followed by labeling autoantibodies attached to the antigen is a strategy for detection of variant forms of the antigen that may not be detectable with conventional sandwich assays, which would only recognize wild type forms of the antigen.

In one embodiment, the second binding agent is an anti-human Ig antibody. For example, the anti-human Ig antibody is selected from the group consisting of: anti-human IgG, anti-human IgM, anti-human IgA, anti-human IgE, anti-human IgD, and mixtures thereof.

In one embodiment, the detection probe further comprises a label. In a particular embodiment, the label is a nanoparticle conjugated to the second binding agent. The nanoparticle may be conjugated directly or indirectly to the second binding agent. For instance, the nanoparticle and second binding agent may each be conjugated to biotin and the nanoparticle and second binding agent may then be joined by an avidin or streptavidin bridge. In one embodiment, the nanoparticle is made of a noble metal, e.g., gold or silver. In one embodiment, the detection probe comprises a fluorophore, a phosphor, a quantum dot, or an enzyme conjugate.

In one embodiment, the first binding agent is bound to a substrate. For example, the substrate may be a nanoparticle, a thin film, or a magnetic bead. In one embodiment, the substrate has a planar surface. In illustrative embodiments, the substrate is made of glass, quartz, ceramic, or plastic. In some embodiments, the substrate is addressable.

In various embodiments, the sample is contacted with the detection probe before, after or simultaneously to contacting with the substrate having the capture probe bound thereto. In one embodiment, the sample is first contacted with the detection probe and then contacted with the capture probe. In another embodiment, the sample is first contacted with the capture probe and then contacted with the detection probe. In yet another embodiment, the sample, the detection probe, and the capture probe are contacted simultaneously.

The formation of a sandwich complex may be detected by various means. For example, the complex may be detected by photonic, electronic, acoustic, opto-acoustic, gravity, electro-chemical, electro-optic, mass-spectrometric, enzymatic, chemical, biochemical, or physical means. In one embodiment, the detecting comprises contacting the substrate with silver stain. In one embodiment, the detecting comprises observing light scattered by the nanoparticles.

In an illustrative embodiment, the disclosure provides a method for the diagnosis of rheumatoid arthritis in a subject comprising: (a) providing a substrate having a capture probe bound thereto, wherein the capture probe comprises one or more antibodies that bind human filaggrin; (b) contacting the substrate with (i) a sample from the subject and (ii) a detection probe under conditions that are suitable for the formation of a complex of the capture probe with filaggrin, and the detection probe with AAs to human fillaggrin, if present in the sample, wherein the detection probe comprises a nanoparticle and an antibody that binds the AAs to human fillaggrin present in the serum of subjects suffering from RA; and (c) detecting the formation of the complex of the capture probe with filaggrin, and the detection probe with the AAs, wherein the presence of the complex is indicative of RA in the subject.

In one embodiment, the capture probe comprises a polyclonal antibody raised against a human filaggrin immunogen. In turn, the polyclonal antibody raised against the filaggrin immunogen may recognize one or more haplotypes, mutant forms, or variants of human filaggrin. Moreover, the polyclonal antibody raised against the filaggrin immunogen may recognize proteins that share the same or similar epitopes, e.g., a histone protein or variant thereof.

In another illustrative embodiment, the disclosure provides a method for the diagnosis of cancer in a subject comprising: (a) providing a substrate having a capture probe bound thereto, wherein the capture probe comprises one or more antibodies that bind p53; (b) contacting the substrate with (i) a sample from the subject and (ii) a detection probe under conditions that are suitable for the formation of a complex of the capture probe with p53, and the detection probe with AAs to p53, if present in the sample, wherein the detection probe comprises a nanoparticle and an antibody that binds the AAs to p53 present in the serum of subjects; and (c) detecting the formation of the complex of the capture probe with p53, and the detection probe with the AAs, wherein the presence of the complex is indicative of cancer in the subject.

In another aspect, the disclosure further relates to diagnostic tests for disease based on a capture probe on a solid phase directed against a conserved region of a disease-associated antigen (i.e. a marker protein or molecule of interest). A patient sample is exposed to the solid phase such that circulating variants of the autoantigen (“neopeptides”) and the cognate neopeptide AA are captured. Alternatively, circulating neopeptide-AA immune complexes are captured by the capture antibody; and the bound complexes are then detected by a sensitive anti-human immunoglobulin-based nanoparticle detection system or using a standard ELISA or immuno-precipitation assay. The capture probe may include, but is not limited to, an antibody or an antigen binding fragment thereof, an aptamer, or specific binding partner (ligand).

In another aspect, the disclosure further relates to diagnostic assays for disease based on capture probes directed against an unknown set of antigens associated with a disease. In this embodiment, proteins are isolated from diseased tissues and are used to immunize animals. The antibodies generated by the immunization process are isolated and deposited onto a solid phase as capture probes. As described above in other embodiments, a sample from the subject is exposed to the solid phase such that disease-associated antigen(s) (“neopeptides”) and the cognate AA are captured and detected.

In another aspect, the disclosure relates to a method for diagnosing or monitoring a disease or medical condition associated with autoantibodies in a subject, the method comprising: (a) measuring the level of a disease-associated antigen in a sample from the subject; (b) measuring the level of disease-associated autoantibodies in the sample; and (c) comparing the levels of the disease-associated antigen and disease-associated autoantibodies in the sample to reference levels of the disease-associated antigen and disease-associated autoantibodies, wherein the presence or stage of a disease or medical condition is indicated by a difference between the reference levels and the levels of the disease-associated antigen and disease-associated autoantibodies in the sample.

In one embodiment, measuring the level of the disease-associated antigen is by contacting the sample with (i) a first capture probe bound to a substrate, wherein the first capture probe comprises a first binding agent capable of specifically binding to a disease-associated antigen and (ii) a first detection probe comprising a second binding agent capable of specifically binding to the disease-associated antigen.

In one embodiment, measuring the level of the disease-associated autoantibody is by contacting the sample with (i) a second capture probe bound to a substrate, wherein the second capture probe comprises a third binding agent capable of specifically binding to a disease-associated autoantibody and (ii) a second detection probe comprising a fourth binding agent capable of specifically binding to the disease-associated autoantibody.

In one embodiment, the first binding agent is an antibody raised against the disease-associated antigen. In one embodiment, the second binding agent is an antibody raised against the disease-associated antigen, and wherein the first binding agent and the second binding agent may be the same or different. In one embodiment, the third binding agent is the disease-associated antigen, and the fourth binding agent is an anti-human Ig antibody. In illustrative embodiments, the anti-human Ig antibody is selected from the group consisting of: anti-human IgG, anti-human IgM, anti-human IgA, anti-human IgE, anti-human IgD, or subtypes and mixtures thereof.

In one embodiment, the reference levels are the level of the disease-associated autoantibodies and the level of the disease-associated antigen in a control population of subjects unaffected by the disease or medical condition. For instance, the presence or stage of the disease or medical condition may be shown by: (i) a similarity between the level of the disease-associated antigen compared to the reference level and (ii) an increase in the level of the disease-associated autoantibodies compared to the reference level.

In an illustrative embodiment, the first binding agent is p53, the second binding agent is a x-p53 antibody, the third binding agent is a x-p53 antibody, and the fourth binding agent is an anti-human Ig antibody. In this embodiment, a similarity between the level of p53 antigen compared to the reference level and an increase in the level of p53 autoantibodies compared to the reference level indicates the presence or stage of cancer. For instance, the cancer is selected from the group consisting of: prostate, breast, colon, lung cancer, and cervical cancer.

In another aspect, the disclosure provides algorithms for the diagnosis, prediction, and/or staging of disease. In one embodiment, the disclosure provides a method for predicting whether a subject has a specific disease or to determine the stage of disease, comprising the steps of: (a) measuring the level of at least two biomarkers selected from the group consisting of: (i) one or more disease-associated autoantibodies, (ii) one or more disease-associated antigens, and (iii) one or more autoantibody-antigen complexes in a sample obtained from the subject; (b) plotting in multidimensional space the levels of the biomarkers from the sample and the levels of the biomarkers in one or more reference standards, wherein each dimension of the multidimensional space corresponds to the level of a single biomarker; and (c) partitioning the plotted levels of the biomarkers from the sample and the one or more reference standards to determine whether the subject has a specific disease or to determine the stage of disease. In one embodiment, the partitioning is by performing a receiver operating characteristic (ROC) analysis. In another embodiment, the partitioning is by performing a CART, CRT, or CHAID analysis.

In one embodiment, the measuring the level of at least two biomarkers comprises measuring the level of autoantibody-antigen complexes with multiple capture probes or detection probes. In one embodiment, the multiple capture probes include two different antibodies that bind to separate epitopes of the same antigen. In one embodiment, the multiple detection probes include different anti-human Ig antibodies or mixtures thereof.

### BRIEF DESCRIPTION OF THE DRAWINGS

FIG. 1. is a schematic diagram showing an illustrative embodiment of the autoantibody fishing assays of the invention.

FIG. 2 is a schematic diagram showing an illustrative embodiment of the autoantibody fishing assays of the invention.

FIG. 3 is a chart showing mean intensities of RA and normal control sera collected using the filaggrin autoantibody assay of the Examples.

FIG. 4 is a chart showing a receiver operating characteristic (ROC) curve based on the filaggrin autoantibody assay of the Examples.

FIG. 5 is a chart showing the correlation of signal detected for p53 autoantibodies (x-axis) and p53 antigen (y-axis) from 50 samples from patients characterized to have cancer (blue circles) and 50 samples obtained from normal patients (green circles).

FIG. 6A is a chart showing the results of an exemplary autoantibody fishing assay for p53 antigen-autoantibody complexes. The three axes of the graph show signal intensity from two x-p53 antibody captures (TF_DO-1 and TF_DO-12) and signal intensity from the antibody capture DO-12 labeled with a different mixture of x-human Ig antibodies (labeled Afx_DO-12).

FIG. 6B is a chart showing the results of an exemplary autoantibody fishing assay for p53 antigen-autoantibody complexes. This plot shows a cross section of FIG. 6A, where signals from two different x-p53 antibodies are used to distinguish cancer patients from normal patients.

FIG. 6C is a chart showing the results of an exemplary autoantibody fishing assay for p53 antigen-autoantibody complexes. This plot shows a cross section of FIG. 6A, where signals from two different mixtures of x-immunoglobulins are used to label p53-autoantibody complexes bound to x-p53 antibody DO-12.

### DETAILED DESCRIPTION

Disease-associated marker proteins may be found both in the tissues and in the bodily fluids of an individual who suffers from a disease or medical condition. Their levels are very low at the early stages of the disease process and increases during progression of the disease. The detection of these proteins has advantageously been used in tests for the diagnosis of cancer but, unfortunately, these assays have many limitations. In particular, commercial antibodies available for use in standard tests are usually not sensitive enough to detect the low levels of disease-associated proteins that are found at the very early stages of the disease, for example in asymptomatic patients, when a treatment would be the most effective. In addition, the genetic mutations or altered post-transcriptional or post-translational processing may be different among different individuals. Most commercial antibodies are not specific for modified forms of disease-associated markers and cross-react with wild-type forms of these proteins, and as a consequence, these antibodies are only useful for detecting wild type forms of the antigen or limited variants. Thus they are only useful for detecting substantial increases in serum levels of wild type forms of single marker proteins, which usually occur at advanced stages of disease.

AAs produced by patients suffering from certain diseases specifically recognize disease-associated marker proteins and variants of the proteins, which broadens the scope of protein isoforms and variants that may be detected. The detection of AAs produced by patients with disease may therefore be used to design alternative, more reliable and sensitive tests to detect the disease condition in an individual from the very beginning of their occurrence.

In the description that follows, a number of terms are utilized extensively. Definitions are herein provided to facilitate understanding of the invention. The terms described below are more fully defined by reference to the specification as a whole. In practicing the invention, many conventional techniques in molecular biology, protein biochemistry, cell biology, immunology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y. (1989)); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press (1986)); Perbal, A Practical Guide to Molecular Cloning; the series, Meth. Enzymol., (Academic Press, Inc. (1984)); Gene Transfer Vectors for Mammalian Cells, Miller & Calos, Eds. (Cold Spring Harbor Laboratory, NY (1987); and Meth. Enzymol., Vols. 154 and 155, Wu & Grossman, and Wu, Eds., respectively. Units, prefixes, and symbols may be denoted in their accepted SI form.

Unless defined otherwise, all technical and scientific terms used herein generally have the same meaning as commonly understood by one of ordinary skill in the art to which this invention belongs. As used in this specification and the appended claims, the singular forms “a,” “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well known and commonly employed in the art.

As used herein, the term “array” refers to a population of different molecules (e.g., capture probes) that are attached to one or more substrates such that the different probe molecules can be differentiated from each other according to relative location. An array can include different probe molecules that are each located at a different addressable location on a substrate. Alternatively, an array can include separate substrates each bearing a different probe molecule. Probes attached to separate substrates can be identified according to the locations of the substrates on a surface to which the substrates are associated or according to the locations of the substrates in a liquid. As used herein, the term “addressable array” or “addressable substrate” refers to an array wherein the individual elements have precisely defined coordinates, so that a given element at a particular position in the array can be identified.

The term “antigen” refers to is a substance that prompts the generation of antibodies and can cause an immune response. As used herein, the term “disease-associated antigen” is a protein or complex of proteins (and sometimes DNA or RNA) that is recognized by AAs present in a biological sample. Examples of disease-associated antigens include, but are not limited to, filaggrin or variants thereof, and p53 or variants thereof. The term “disease-associated antigen” also includes other antigens that are immunologically cross-reactive with AAs present in a sample.

As used herein, the term “antibody” means a polypeptide comprising a framework region from an immunoglobulin gene or fragments thereof that specifically binds and recognizes an antigen, e.g., a disease-associated antigen. Use of the term antibody is meant to include whole antibodies, including single-chain whole antibodies, antibody fragments such as Fab fragments, and other antigen-binding fragments thereof. The term “antibody” includes bispecific antibodies and multispecific antibodies so long as they exhibit the desired biological activity or function.

An “autoantibody” (abbreviated “AA”) is an antibody produced by the immune system of a subject that is directed against one or more of the subject's own proteins.

As used herein, the term “binding agent” is intended to mean a compound, a macromolecule, including polypeptide, DNA, RNA and carbohydrate that selectively binds a target molecule. For example, a binding agent can be a polypeptide that selectively binds with high affinity or avidity to a target analyte without substantial cross-reactivity with other polypeptides that are unrelated to the target analyte. The affinity of a binding agent that selectively binds a target analyte will generally be greater than about 10⁻⁵ M, such as greater than about 10⁻⁶ M, including greater than about 10⁻⁸ M and greater than about 10⁻⁹ M. Specific examples of such selective binding agents include a polyclonal or monoclonal antibody specific for a disease-associated antigen or human immunoglobulin. For certain applications, a binding agent can be used that preferentially recognizes a particular haplotype or variant of the disease-associated antigen. The binding agent can be labeled with a detectable moiety, if desired, or rendered detectable by specific binding to a detectable secondary binding agent.

As used herein, the term “capture probe” refers to a molecule capable of binding to a target analyte, e.g., a disease-associated antigen. One example of a capture probe includes antibodies that recognize autoantigens present in a biological sample from patients having or suspected of having a disease, e.g., rheumatoid arthritis or cancer. Other examples of capture probes include aptamers, protein ligands, etc., which are described for instance, in PCT/US01/10071 (Nanosphere, Inc.).

The term “conserved region” refers to a region in a nucleotide or amino acid sequence that exhibits a high degree of sequence homology among all of the sequences of interest, e.g., all variants or haplotypes of a gene or protein. In the present context, a conserved region exhibits a high degree of sequence identity over at least 10 base pairs (bp)/3 amino acids (a.a), at least 20 bp/7 a.a., or at least 30 bp/10 a.a.

As used herein, the term “complex” means an aggregate of two or more molecules that results from specific binding between the molecules, such as an antibody and an antigen, a receptor and a ligand, etc.

A “detection probe” is a labeled molecule including one or more binding agents, wherein the one or more binding agents specifically bind to a specific target analyte. The label itself may serve as a carrier, or the probe may be modified to include a carrier. Carriers that are suitable for the methods include, but are not limited to, nanoparticles, quantum dots, dendrimers, semi-conductors, beads, up- or down-converting phosphors, large proteins, lipids, carbohydrates, or any suitable inorganic or organic molecule of sufficient size, or a combination thereof.

As used herein, the term “disease-associated antigen”, refers to a substance associated with a disease or medical condition in a subject, which causes an autoimmune response in that subject, resulting in the production of AAs. Disease-associated antigens include the wild-type protein, complexes, and aggregates as well as modified forms (mutants, haplotypes, or other variant forms), complexes, and aggregates of wild-type proteins.

The term “haplotype” as used herein is intended to refer to a set of alleles that are inherited together as a group (are in linkage disequilibrium) at statistically significant levels (ρ_{corr}<0.05). In the context of the present invention, a haplotype preferably refers to a combination of biallelic marker alleles found in a given individual and which may be associated with a phenotype.

The term “homology” refers to sequence similarity between two peptides or between two nucleic acid molecules. Homology may be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. “Identity” means the degree of sequence relatedness between polypeptide or polynucleotide sequences, as the case may be, as determined by the match between strings of such sequences. “Identity” and “homology” can be readily calculated by known methods. Suitable computer program methods to determine identity and homology between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215: 403-410 (1990). The BLAST X program is publicly available from NCBI and other sources (BLAST Manual, Altschul, S., et al, NCBI NLM NIH Bethesda, Md. 20894; Altschul, S., et al., J. Mol. Biol. 215: 403-410 (1990).

As used herein, the terms “immunologically cross-reactive” and “immunologically-reactive” are used interchangeably to mean an antigen which is specifically reactive with an antibody which was generated using the same (“immunologically-reactive”) or different (“immunologically cross-reactive”) antigen.

As used herein, the term “immunologically-reactive conditions” means conditions which allow an antibody to bind to that epitope or a structurally similar epitope to a detectably greater degree than the antibody binds to substantially all other epitopes, generally at least two times above background binding, preferably at least five times above background. Immunologically-reactive conditions are dependent upon the format of the antibody binding reaction and typically are those utilized in immunoassay protocols. See, Harlow & Lane, Antibodies, A Laboratory Manual (Cold Spring Harbor Publications, New York (1988), for a description of immunoassay formats and conditions.

As used herein, the terms “label” or “detectable label” refers to a marker that may be detected by photonic, electronic, opto-electronic, magnetic, gravitic, acoustic, enzymatic, magnetic, paramagnetic, or other physical or chemical means. The term “labeled” refers to incorporation of such a detectable marker, e.g., by incorporation of a radiolabeled molecule or attachment to a nanoparticle.

As used herein, the term “level” is intended to mean the amount, accumulation or rate of synthesis of a molecule. The term can be used to refer to an absolute amount of a molecule in a sample or to a relative amount of the molecule, including amounts determined under steady-state or non-steady-state conditions. The level of a molecule can be determined relative to a control molecule in a sample. The level of a molecule also can be referred to as an expression level.

As used herein, the term “medical condition” includes, but is not limited to, any condition, disease, or disorder manifested as one or more physical and/or psychological symptoms for which treatment and/or prevention is desirable, and includes previously and newly identified diseases and other disorders. For example, a medical condition may be rheumatoid arthritis or cancer.

The term “monoclonal antibody” as used herein refers to an antibody obtained from a population of substantially homogeneous antibodies, i.e., the individual antibodies comprising the population are identical except for possible naturally occurring mutations that may be present in minor amounts. For example, a monoclonal antibody can be an antibody that is derived from a single clone, including any eukaryotic, prokaryotic, or phage clone, and not the method by which it is produced. A monoclonal antibody composition displays a single binding specificity and affinity for a particular epitope. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. Monoclonal antibodies can be prepared using a wide variety of techniques known in the art including, e.g., but not limited to, hybridoma, recombinant, and phage display technologies. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The monoclonal antibodies may also be isolated from phage antibody libraries using the techniques described in Clackson et al., Nature 352:624-628 (1991) and Marks et al., J. Mol. Biol. 222:581-597 (1991), for example.

The term “ortholog” refers to genes or proteins which are homologs via speciation, e.g., closely related and assumed to have common descent based on structural and functional considerations. Orthologous proteins function as recognizably the same activity in different species. The term “paralog” denotes a polypeptide or protein obtained from a given species that has homology to a distinct polypeptide or protein from that same species.

As used herein, the term “polyclonal antibody” means a preparation of antibodies derived from at least two (2) different antibody-producing cell lines. The use of this term includes preparations of at least two (2) antibodies that contain antibodies that specifically bind to different epitopes or regions of an antigen.

As used herein, the terms “polypeptide,” “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature.

As used herein, the term “reference level” is intended to mean a control level of a biomarker, e.g., disease-associated autoantibody, used to evaluate a test level of the biomarker in a sample from an individual. A reference level can be a normal reference level or a disease-state reference level. A normal reference level is an amount of expression of a biomarker in a non-diseased subject or subjects. A disease-state reference level is an amount of expression of a biomarker in a subject with a positive diagnosis for the disease or condition. A reference level also can be a stage-specific reference level. A stage-specific reference level refers to a level of a biomarker characteristic of a given stage of progression of a disease or condition.

As used herein, the term “sample” means sample material derived from or contacted by living cells. The term “sample” is intended to include tissues, cells and biological fluids isolated from a subject, as well as tissues, cells and fluids present within a subject. Biological samples include, e.g., but are not limited to, whole blood, plasma, serum, semen, cell lysates, saliva, tears, urine, fecal material, sweat, buccal, skin, cerebrospinal fluid, and hair. Biological samples can also be obtained from biopsies of internal organs. Biological samples can be obtained from subjects for diagnosis or research or can be obtained from undiseased individuals, as controls or for basic research.

The term “specific binding” refers to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10⁻⁶ M. In other embodiments, the antigen and antibody will bind with affinities of at least 10⁻⁷ M, 10⁻⁸ M to 10⁻⁹ M, 10⁻¹⁰ M, 10⁻¹¹ M, or 10⁻¹² M.

As used herein the phrase “splice variant” refers to mRNA molecules produced from primary RNA transcripts that have undergone alternative RNA splicing. Alternative RNA splicing occurs when a primary RNA transcript undergoes splicing, generally for the removal of introns, which results in the production of more than one mRNA molecule each of which may encode different amino acid sequences. The term “splice variant” also refers to the proteins encoded by the above mRNA molecules.

As used herein, the term “subject” means the subject is a mammal, such as a human, but can also be an animal, e.g., domestic animals (e.g., dogs, cats and the like), farm animals (e.g., cows, sheep, pigs, horses and the like) and laboratory animals (e.g., monkey, rats, mice, rabbits, guinea pigs and the like).

As used herein, the term “substitution” is one of mutations that is generally used in the art. Substitution variants have at least one amino acid residue in a polypeptide molecule replaced by a different residue. “Conservative substitutions” typically provide similar biological activity as the unmodified polypeptide sequence from which the conservatively modified variant was derived. Conservative substitutions typically include the substitution of one amino acid for another with similar characteristics. Conservative substitution tables providing functionally similar amino acids are well known in the art. For example, the following six groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containg: Methionine (M), Cysteine (C); Basic (Cationic): Arginine (R), Lysine (K), Histidine (H); Acidic (Anionic): Aspartic acid (D), Glutamic acid (E); Amide: Asparagine (N), Glutamine (Q).

As used herein, the term “substrate” refers to any surface capable of having capture probes bound thereto. Such surfaces include, but are not limited to, glass, metal, plastic, or materials coated with a functional group designed for binding of capture probes or analytes.

As used herein, the terms “treating” or “treatment” or “alleviation” refers to both therapeutic treatment and prophylactic or preventative measures, wherein the object is to prevent or slow down (lessen) the targeted pathologic condition or disorder. A subject is successfully “treated” for a disorder characterized by increased autoantibody levels if the subject shows observable and/or measurable reduction in or absence of one or more signs and symptoms of a particular disease or condition. For example, for cancer, reduction in the number of cancer cells or absence of the cancer cells; reduction in the tumor size; inhibition (i.e., slow to some extent and preferably stop) of tumor metastasis; inhibition, to some extent, of tumor growth; increase in length of remission, and/or relief to some extent, one or more of the symptoms associated with the specific cancer; reduced morbidity and mortality, and improvement in quality of life issues.

As used herein, the term “variant polypeptide” refers to a polypeptide that differs from a naturally occurring polypeptide in amino acid sequence or in ways that do not involve amino acid sequence modifications, or both. Non-sequence modifications include, but are not limited to, changes in acetylation, methylation, phosphorylation, carboxylation, or glycosylation. Variants may also include sequences that differ from the wild-type sequence by one or more amino acid substitutions, deletions, or insertions. The term “allelic variant” denotes any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

### Disease-Associated Antigens and Autoantibodies

The development of immunologic responsiveness to self is called autoimmunity and reflects the impairment of self-tolerance. Immunologic, environmental, and genetic factors are closely interrelated in the pathogenesis of autoimmunity. The frequency of autoimmune antibodies (AAs) in the general population increases with age, suggesting a breakdown of self-tolerance with aging. AAs also may develop as an aftermath of disease tissue damage.

The development of autoimmunity may involve the breakdown or circumvention of self-tolerance. The potential for the development of AAs probably exists in most individuals. For example, normal human B cells are capable of reacting with several self-antigens, but are suppressed from producing AAs by one or more tolerance mechanisms. Precommitted B cells in tolerant individuals can be stimulated in several ways. For example, tolerance involving only T cells, induced by persistent low levels of circulating self-antigens, may breakdown in the presence of substances such as endotoxin. Such substances stimulate the B cells directly to produce AAs. Another tolerance mechanism involves suppressor T cells. A decrease in suppressor T cell activity therefore may also lead to production of AAs.

In various embodiments, the methods described herein may be used to detect AAs raised against disease-associated antigens. Disease-associated antigens and AAs have been detected in patients suffering from a variety of diseases or conditions, including but not limited to, autoimmune disease and cancer. For instance, the disease-associated antigen may be a variant form of a polypeptide, i.e., a polypeptide formed as the result of mutation. Such variants are also referred to herein as “neopeptides.”

In one embodiment, the methods described herein may be used to detect AAs associated with autoimmune disease. The autoantigens may be specifically expressed in the diseased tissue or may be expressed systemically in the subject. Autoimmune disorders include, but are not limited to: systemic lupus erythematosus (SLE), Rheumatoid arthritis (RA), Juvenile Rheumatoid arthritis (JRA), acute disseminated encephalomyelitis, Addison's disease, ankylosing spondylitis, antiphospholipid antibody syndrome, autoimmune hemolytic anemia, autoimmune hepatitis, autoimmune inner ear disease, bullous pemphigoid, celiac disease, Chagas disease, chronic obstructive pulmonary disease, dermatomyositis, diabetes mellitus type 1, endometriosis, Goodpasture's syndrome, Graves' disease, Guillain-Barre syndrome, Hashimoto's disease, Hidradenitis supprativa, idiopathic thrombocytopenic purpura, interstitial cystitis, morphea, multiple sclerosis, myasthenia gravis, neuromyotonia, pemphigus vulgaris, pernicious anemia, polymyositis, primary biliary cirrhosis, schleroderma, Sjogren's syndrome, temporal arteritis, vasculitis, vitiligo, Wegener's granulomatosis, Progressive systemic sclerosis, Polyarteritis nodosa, Behcets disease, Ankylosing spondylitis, Reiter's syndrome, Psoriatic arthritis, Relapsing polychondritis, Weber-Christian disease, Collagen vascular diseases, and Hypogammaglobulinemia. See also Fudenberg et al., Basic and Clinical Immunology, 2nd Ed., 1978, Lange.

In one example, SLE is characterized by an array of AAs to cell and tissue antigens. Among the different autoantigenic candidates that are recognized by AAs in SLE, two nuclear antigens that are considered pathognomonic of SLE, double-stranded DNA (dsDNA) and the Sm antigens of the U-1 small nuclear ribonucleoprotein complex. Antibodies to these autoantigens are sufficiently discriminating to be part of the American College of Rheumatology (ACR) classification criteria for SLE. In addition, antibodies to phospholipids are included in the ACR criteria, although they are less specific for the disease.

In another example, Grave's disease is an autoimmune disease caused by antibody and T-cell responses to epitopes on thyroid-stimulating receptor (TSHR). Likewise, human acetylcholine receptor (AChR) AAs have been associated with myasthenia gravis (MG). Other antigens that may stimulate production of AAs in subjects suffering from autoimmune diseases, such as RA, include, but are not limited to filaggrin; nuclear, nucleolar or cytoplasmic autoantigens which consist of nucleic acids or nucleic acid-protein complexes; chromatin; C1q; Citrullinated antigens; Fibrinogen; Fibrin; Vimentin; Alpha-enolase; Perinuclear factor; and Keratin.

In one embodiment, the methods described herein may be used to detect AAs associated with RA. A number of autoantigens for RA have been described in the literature. See Blass et al., The Immunologic Homunculus in Rheumatoid Arthritis. Arthritis and Rheumatism 1999; 42:2499-2506. Some RA autoantigens are well characterized biochemically and by their antigenic character. For example, the Sa and filaggrin antigens are antigens that are not present in the inflamed joint as such, but draw attention as targets of very disease-specific immune responses. The Sa antigen is a 50 k protein isolated from human spleen or placenta. Sa-specific antibodies occur in RA patients with a 43% sensitivity and a 78% to 99% specificity. Filaggrin is a 42 k protein involved in the crosslinking of intermediate filament proteins, namely, cytokeratin, and is present in the endothelium. Antibodies to filaggrin seem to be identical to previously described antiperinuclear factor and antikeratin antibodies. The major determinant of the epitope(s) targeted by antifilaggrin antibodies is citrulline, a modified arginine residue. Specific examples of AAs that have been associated with RA include, but are not limited to rheumatoid factors (RFs), antibodies to citrullinated antigens such as fillagrin and anti-CCP antibody, and antibodies to immunoglobulin binding protein (BiP).

In one embodiment, the detection methods can be used to detect the presence, absence, and/or amount of anti-filaggrin antibodies (AFA) in a biological sample. Elevated AFAs can be found in patients who have a negative RF, the classic test for RA. In some embodiments, the AFAs may specifically bind one or more filaggrin variants, including citrullinated forms of the polypeptide or genetic mutations. The filaggrin gene (FLG) is located within the epidermal differentiation complex (EDC) on 1q21.3, a gene cluster expressed late in epidermal differentiation. FLG contains a large and highly repetitive exon 3, which also shows population size variation (12.7-14.7 kb). This exon encodes 10-12 full tandem repeats of the filaggrin protein that are almost 100% identical at the DNA sequence level, flanked by two partial repeats. A number of filaggrin variants have been identified as associated with certain diseases. For example, three loss-of-function variants of the filaggrin gene have been discovered: R501X, 2282del4 and 3702del1. In some RA patients, heterozygous carriers of either of the these FLG variants exhibited a significantly elevated prevalence of AAs to citrullinated peptides (CCP-2) (80%) compared to non-carriers (51.9%) (Huffmeier et al., Ann Rheum Dis 2008; 67:131-133.

In one embodiment, the detection of AFAs in a sample is indicative of atopic dermatitis or ichthyosis vulgaris in the subject. A strong association between the occurrence of atopic dermatitis or ichthyosis vulgaris and 15 filaggrin variants was established (Sandilands et al., Nature Genetics 2007; 39: 650-654). These variants include: R501X, 2282del4, 2702delG, R1474X, 5360delG, 6687delAG, E2422X, 7267delCA, R2477X, S3247X, 11029delCA, 11033del4, Q3683X, 3321delA, and S2554X. Some of these variants also show a strong association with moderate-to-severe childhood eczema. The data described in these studies suggest that haplotypes of the filaggrin gene consist of both prevalent and rare risk alleles.

In one embodiment, the methods described herein may be used to detect AAs associated with cancer. Cancer is a disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. See National Cancer Institute website (U.S. National Institutes of Health). For example, AAs directed against p53, Ras, c-myc, MUC-1, c-erbβ2, or PSA proteins may be detected in cancer patients. See Soussi, Canc Res (2006) 60, 1777-88.

In one embodiment, the methods described herein may be used to detect AAs raised against tumor associated antigens. In the context of the present invention, “tumor associated antigen” refer to antigens that are common to specific hyperproliferative disorders. In certain aspects, the tumor-associated antigens are derived from cancers including but not limited to primary or metastatic melanoma, thymoma, lymphoma, sarcoma, lung cancer, gastic cancer, liver cancer, non-Hodgkin's lymphoma, Hodgkin's lymphoma, leukemia, uterine cancer, cervical cancer, bladder cancer, kidney cancer and adenocarcinomas such as breast cancer, prostate cancer, ovarian cancer, pancreatic cancer, and the like. Tumor-associated antigens that may stimulate production of AAs in subjects suffering from cancer include, but are not limited to, an overexpressed tumor-associated antigen, a testis-tumor antigen, a mutated tumor-associated antigen, a differentiation tumor-associated antigen tyrosinase, MART, trp, MAGE-1, MAGE-2, MAGE-3, gp100, HER-2, Ras, PSA BCR-ABL, CASP, CDK, Ras, p53, HER-2/neu, CEA, MUC, TW1, PAP, survivin, telomerase, EGFR, PSMA, PSA, PSCA, tyrosinase, MART, TRP, gp100, MART, MAGE, BAGE, GAGE, LAGE/NY-ESO, RAGE, SSX-2, CD19, CD20, 5-alpha-reductase; prostasomes; glucose-regulated 78-kDa protein (GRP78); MUC1; PARIS-1; c-myc; c-erbβ2; Cytokine-inducible serine/threonine-protein kinase; Interleukin 1, beta; Nitric oxide synthase 1 neuronal); Tumor suppressor p53-binding protein 2; Myc proto-oncogene protein; Multi PDZ domain protein MUPP1; Diaphanous protein homolog 1; DNA-repair protein complementing XP-A cells; Citron (rho-interacting, serine/threonine kinase 21); TIA1 cytotoxic granule-associated RNA binding protein-like 1; Transcription factor E2F2; DNA polymerase epsilon, catalytic subunit A; Chondroitin sulfate proteoglycan 5 (neuroglycan C); DEAD (Asp-Glu-Ala-Asp) box polypeptide 1; BCL2-like 1; Ribosomal protein S6 kinase, 70 kDa, polypeptide 1; Calcium/calmodulin-dependent protein kinase kinase 1, alpha; Sjögren's syndrome antigen B (autoantigen La); Junction plakoglobin; Calnexin; Protein tyrosine phosphatase, receptor-type, Z polypeptide 1; Scavenger receptor class B, member 1; Neurexin 1-alpha precursor; Transcription initiation factor IIB; Neutrophil cytosol factor 2; Microtubule-associated protein tau; Lymphocyte cytosolic protein 2; Far upstream element (FUSE) binding protein 1; Protein kinase, cAMP-dependent, regulatory, type II, beta; Protein kinase C, alpha; Mitogen-activated protein kinase 1; Katanin p80 (WD repeat containing) subunit B1; Tumor necrosis factor (TNF superfamily, member 2); Interferon-induced, double-stranded RNA-activated protein kinase; Gephyrin; Protein kinase C, eta; Optineurin; BCL2-associated X protein; Phospholipase C, beta 1 (phosphoinositide-specific); Diacylglycerol kinase, theta; CDC25C; Caspase 4, apoptosis-related cysteine protease; Cellular tumor antigen p53; Non-POU domain containing, octamer-binding; Doublecortin; CrmA, serine proteinase inhibitor 2; Amyloid beta A4 precursor protein-binding family A member 3; G1/S-specific cyclin D3, and combinations thereof.

In a particular embodiment, AAs directed against p53 neopeptides are detected. Assays for p53 neopeptides and/or AAs may be useful for cancer screening, early diagnosis/identification of latent disease, treatment planning, treatment monitoring, prognosis for cancer progression, prognosis for recurrence, and prognosis for metastasis. The AAs may be formed due to the overproduction of p53 or to the emergence of immunogenic mutant neopeptides, or both. In one embodiment, the methods provide for the detection of AAs raised against p53 neopeptides—and not to native p53. For instance, the methods detect the AAs associated with neopeptide forms because binding agents of the capture probes are specifically raised against conserved regions of the protein. These regions are then shielded by binding to the solid phase. Consequently, AAs that are bound to the variable regions of the protein are exposed to the solution phase during the assay, thus enabling the detection the anti-neopeptide AAs.

In another embodiment, the methods described herein may be used to detect AAs directed to the neopeptide forms of the polypeptides TAF1B, MACS, UVRAG, ELAVL3, TCF6L1, ABCF1, AIM2, CHD2, FLJ11053, KIAA1052, ACVR2 and HT001. The neopeptide forms of these polypeptides may be frameshift mutations, including, but not limited to: (1) the insertion of one A in the A11 repeats of the genes TAF1B, MACS, HT001, FLJ11053, KIAA1052; (2) the insertion of two A in the A11 repeats of the genes TAF1B, MACS, HT001, FLJ11053, KIAA1052; (3) the deletion of one A in the A11 repeats of the genes TAF1B, MACS, HT001, FLJ11053, KIAA1052; (4) the deletion of two A in the A11 repeats of the genes TAF1B, MACS, HT001, FLJ11053, KIAA1052; (5) the insertion of one A in the A10 repeats of the genes CHD2, UVRAG, TCF6L1, ABCF1, AIM2; (6) the insertion of two A in the A10 repeats of the genes CHD2, UVRAG, TCF6L1, ABCF1, AIM2; (7) the deletion of one A in the A10 repeats of the genes CHD2, UVRAG, TCF6L1, ABCF1, AIM2; (8) the deletion of two A in the A10 repeats of the genes CHD2, UVRAG, TCF6L1, ABCF1, AIM2; (9) the insertion of one A in the A8 repeat of the gene ACVR2; (10) the insertion of two A in the A8 repeat of the gene ACVR2; (11) the deletion of one A in the A8 repeat of the gene ACVR2; (12) the deletion of two A in the A8 repeat of the gene ACVR2; (13) the insertion of one G in the G9 repeat of the gene ELAVL3; or (14) the insertion of two G in the G9 repeat of the gene ELAVL3; (15) the deletion of one G in the G9 repeat of the gene ELAVL3; and (16) the deletion of two G in the G9 repeat of the gene ELAVL3. See U.S. Patent Publication No. 2005/0239070. Any or all of the neopeptides may result in formation in disease-associated AAs. In accordance with the procedures described herein, one of skill in the art could generate binding agents capable of specifically binding to these neopeptides.

In one embodiment, the methods described herein may be used to detect AAs associated with cardiovascular disease. The disease-associated antigen may be a cardiac marker well known in the art. Antigens associated with cardiac disease and which may stimulate production of AAs in subjects suffering from this disease include, but are not limited to, cardiac troponin-I; cardiac troponin-T; cardiac troponin-C; p200-epitope of Ro52; and human cardiac myosin.

Antigens associated with neurodegenerative disorders and which may stimulate production of AAs in patients suffering from these diseases include, but are not limited to phospho tau; amyloid beta; alpha-synuclein; protease-resistant prions; superoxide; dismutase-1, huntingtin, and ataxin.

### Autoantibody Detection Assays

In one aspect, the methods include using a sandwich assay to detect the AAs. Sandwich assays generally involve the use of two binding agents, e.g., antibodies, each capable of binding to a different portion, or epitope, of the protein(s) to be detected and/or quantitated. In a sandwich assay, the analyte is typically bound by a first binding agent which is immobilized on a solid support, and thereafter a second binding agent binds to the analyte, thus forming an insoluble complex. See, e.g., U.S. Pat. No. 4,376,110. Alternatively, the sandwich assay may be performed in solution, also referred to as a homogeneous assay. See, e.g., U.S. Pat. No. 7,413,862.

In some embodiments of these methods, the capture probe including a first binding agent is capable of specifically binding to a disease-associated antigen, e.g., a neopeptide, which is bound to one or more AAs. In turn, the detection probe including a second binding agent binds to the AAs. Thus, a four-part complex is formed between: (1) the capture probe, (2) the disease-associated antigen, (3) the AA, and (4) the detection probe. In an alternative embodiment, the positions of the first and second binding agents are reversed, such that the capture probe attached to the solid support is capable of specifically binding to the AAs and the detection probe is capable of specifically binding to the disease-associated antigen.

The methods can be performed using any immunological technique known to those skilled in the art of immunochemistry. As examples, ELISA, immunofluorescence, radioimmunoassays or similar techniques may be utilized. In general, an appropriate capture probe is immobilized on a solid surface and the sample to be tested (e.g. human serum) is brought into contact with the capture probe. For example, modified glass substrates that covalently or non-covalently bind proteins can be used to bind antibodies. The substrate may be treated with suitable blocking agents to minimize non-specific binding. If the disease-associated antigen is present in the sample, a complex between the disease-associated antigen and the capture probe is formed. A detection probe is then added, which specifically recognizes an epitope of a human immunoglobulin (Ig), if present. The anti-human immunoglobulin detection probe may be directed against the Fc region of the human antibody and with as little cross-reactivity as possible against the capture antibody species.

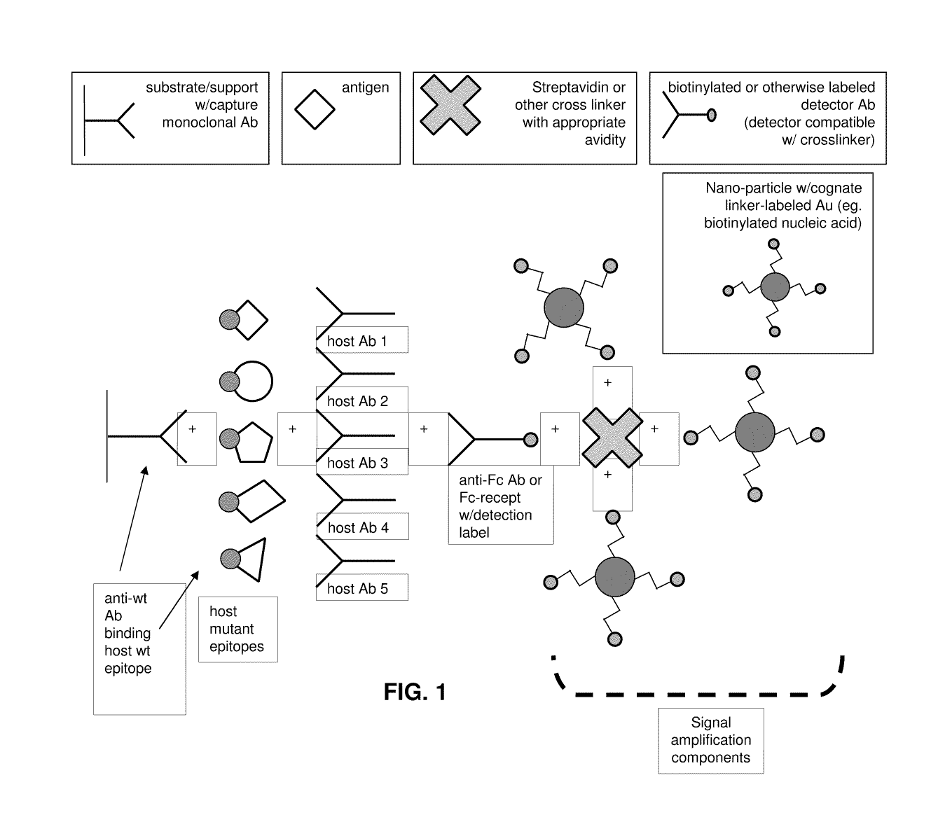

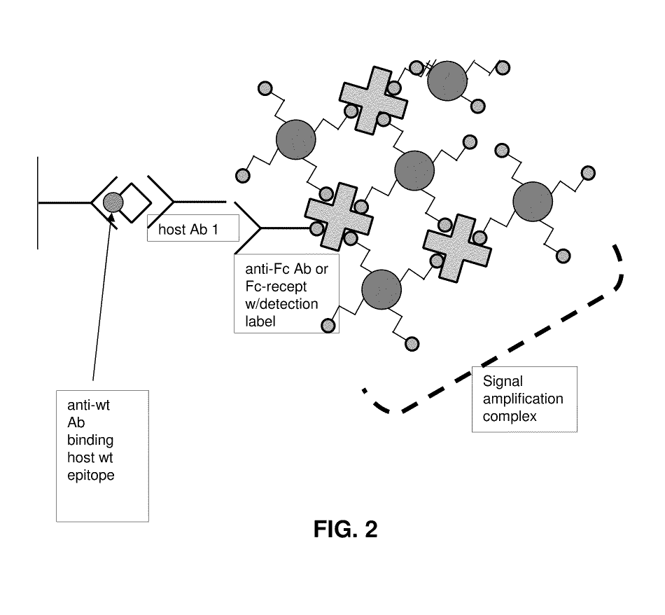

In one embodiment, the methods comprise contacting a sample with a capture probe including a antibody capable of binding to a disease-associated antigen. The sample is also contacted with a detection probe including anti-human Ig antibodies. The presence, absence, and/or amount of the complex may be detected, wherein the presence or absence of the complex is indicative of the presence or absence of the AAs. (See FIG. 1 and FIG. 2).

The complex can then be detected or quantitatively measured. The detection probe may be labeled with biochemical markers such as, for example, a nanoparticle, horseradish peroxidase (HRP) or alkaline phosphatase (AP), and detection of the complex can be achieved by the addition of a substrate for the enzyme which generates a calorimetric, chemiluminescent or fluorescent product. Alternatively, the presence of the complex may be determined by addition of a marker protein labeled with a detectable label, for example an appropriate enzyme. In this case, the amount of enzymatic activity measured is inversely proportional to the quantity of complex formed and a negative control is needed as a reference to determine the presence of antigen in the sample. Another method for detecting the complex may utilize antibodies or antigens that have been labeled with radioisotopes followed by measure of radioactivity.

The sample may be contacted with the detection probe before, after, or simultaneously with the capture probe. In one embodiment, the sample is first contacted with the detection probe so that AAs present in the sample bind to the detection probe to form a target analyte complex. The mixture is then contacted with the substrate having capture probes bound thereto so that the target analyte complex binds to the capture probe on the substrate. In another embodiment, the sample is first contacted with the substrate so that a target analyte complex present in the sample binds to a capture probe, and the target analyte complex bound to the capture probe is then contacted with the detection probe so that the AAs bind to the detection probe. In another embodiment, the sample, the detection probe and the capture probe on the substrate are contacted simultaneously.

In some embodiments, the antigens recognized by AAs, when used in a sandwich assay employing gold-nanoparticle detection with silver enhancement, significantly improves the LOD for auto-antibodies by lowering the detectable concentration of the complex formed between the antigen and the captured antibody. In some embodiments, the assay employs a mixed set of biotinylated secondary antibody isotypes which allow more favorable detection of the response of AAs—particularly a mixture of anti-immunoglobulins, such as anti-IgG, anti-IgM, anti-IgA, anti-IgE and anti-IgD may be used as detection antibodies.

Embodiments of the invention provide a diagnostic method for disease, which involves: (a) assaying the levels AAs by measuring binding of AAs in cells or body fluid of an individual; (b) comparing the amount of AAs with a standard or reference level, whereby an increase or decrease in the assayed AAs compared to the standard level is indicative of a medical condition, e.g., RA or cancer.

In one embodiment, a binding assay refers to an assay format wherein an disease-associated antigen is mixed with a biological sample under conditions suitable for binding between the antigen and AAs in the biological sample, if present. The amount of binding is compared with a suitable control, which can be the amount of binding in the absence of the AAs, the amount of the binding in the presence of a non-specific immunoglobulin composition, or both. In one embodiment, a detection assay for AAs may utilize a polyclonal filaggrin antibody as a capture probe. In another, embodiment, the detection assay for AAs may utilize an anti-p53 antibody.

### Diagnostic Assays Based on Comparison of Disease-Associated Antigen and AA Levels

In one embodiment, the disclosure provides a method for diagnosing or monitoring a disease or medical condition associated with autoantibodies in a subject, the method comprising: (a) measuring the level of the disease-associated antigen in a sample from the subject; (b) measuring the level of the disease-associated AAs in the sample; and (c) comparing the levels of the disease-associated antigen and disease-associated AAs in the sample to reference levels of the disease-associated antigen and disease-associated AAs, wherein the presence or stage of a disease or medical condition is indicated by a difference between the reference levels and the levels of the disease-associated antigen and disease-associated AAs in the sample. Thus, the presence, absence, and/or amount of AAs and antigens may be measured, wherein a correlation between the amount of antigen present and the amount of autoantibody present may be diagnostic or prognostic of a particular disease or medical condition.

Reference Levels. The reference level used for comparison with the measured level for a disease-associated antigen or AA may vary, depending on the aspect of the invention being practiced, as will be understood from the foregoing discussion. For disease diagnostic methods, the “reference level” is typically a predetermined reference level, such as an average of levels obtained from a population that is not afflicted with the disease or medical condition, but in some instances, the reference level can be a mean or median level from a group of individuals including diseased patients. In some instances, the predetermined reference level is derived from (e.g., is the mean or median of) levels obtained from an age-matched population. Alternatively, the reference level may be a historical reference level for the particular patient (e.g., a disease-associated antigen or AA level that was obtained from a sample derived from the same individual, but at an earlier point in time).

For disease staging or stratification methods (i.e., methods of classifying diseased patients into mild, moderate and severe stages of disease), the reference level is normally a predetermined reference level that is the mean or median of levels from a population which has been diagnosed with disease. In some instances, the predetermined reference level is derived from (e.g., is the mean or median of) levels obtained from an age-matched population.

Age-matched populations (from which reference values may be obtained) are ideally the same age as the individual being tested, but approximately age-matched populations are also acceptable. Approximately age-matched populations may be within 1, 2, 3, 4, or 5 years of the age of the individual tested, or may be groups of different ages which encompass the age of the individual being tested. Approximately age-matched populations may be in 2, 3, 4, 5, 6, 7, 8, 9, or 10 year increments (e.g. a “5 year increment” group which serves as the source for reference values for a 62 year old individual might include 58-62 year old individuals, 59-63 year old individuals, 60-64 year old individuals, 61-65 year old individuals, or 62-66 year old individuals).

Comparing Levels of Disease-Associated Antigens and/or AAs. The process of comparing a measured value and a reference value can be carried out in any convenient manner appropriate to the type of measured value and reference value for the disease-associated antigen or AA at issue. Measuring can be performed using quantitative or qualitative measurement techniques, and the mode of comparing a measured value and a reference value can vary depending on the measurement technology employed. For example, when a qualitative assay is used to measure disease-associated antigen or AA levels, the levels may be compared by comparing data from densitometric or spectrometric measurements (e.g., comparing numerical data or graphical data, such as bar charts, derived from the measuring device). However, it is expected that the measured values used in the methods of the invention will most commonly be quantitative values (e.g., quantitative measurements of signal intensity).

A measured value is generally considered to be substantially equal to or greater than a reference value if it is at least 95% of the value of the reference value (e.g., a measured value of 1.71 would be considered substantially equal to a reference value of 1.80). A measured value is considered less than a reference value if the measured value is less than 95% of the reference value (e.g., a measured value of 1.7 would be considered less than a reference value of 1.80). A measured value is considered more than a reference value if the measured value is at least more than 5% greater than the reference value (e.g., a measured value of 1.89 would be considered more than a reference value of 1.80).

The process of comparing may be manual (such as visual inspection by the practitioner of the method) or it may be automated. For example, an assay device may include circuitry and software enabling it to compare a measured value with a reference value for a disease-associated antigen or AA. Alternatively, a separate device (e.g., a digital computer) may be used to compare the measured value(s) and the reference value(s). Automated devices for comparison may include stored reference values for the disease-associated antigen or AA being measured, or they may compare the measured value(s) with reference values that are derived from contemporaneously measured reference samples.

In some embodiments, the methods of the invention utilize “simple” or “binary” comparison between the measured level(s) and the reference level(s) (e.g., the comparison between a measured level and a reference level determines whether the measured level is higher or lower than the reference level). For AA levels, a comparison showing that the measured value for the AA is higher than the reference value indicates or suggests a diagnosis of disease.

In certain aspects, the comparison is performed to determine the magnitude of the difference between the measured and reference values (e.g., comparing the “fold” or percentage difference between the measured value and the reference value). A fold difference that is about equal to or greater than the minimum fold difference disclosed herein suggests or indicates a diagnosis of a disease or medical condition, as appropriate to the particular method being practiced. A fold difference can be determined by measuring the absolute concentration of the disease-associated antigen or AA and comparing that to the absolute value of a reference, or a fold difference can be measured by the relative difference between a reference value and a sample value, where neither value is a measure of absolute concentration, and/or where both values are measured simultaneously.

As will be apparent to those of skill in the art, when replicate measurements are taken for the biomarker(s) tested, the measured value that is compared with the reference value is a value that takes into account the replicate measurements. The replicate measurements may be taken into account by using either the mean or median of the measured values as the “measured value.”

### Multiple Marker Analysis for Subject Rule-In and Rule-Out

While assays using a single capture probe may be informative in the diagnosis of disease, combining the information from two or more capture probes into one diagnostic algorithm can make a substantial improvement in the prediction. By optimizing the combined information, it is possible to increase the specificity and sensitivity of the assay.

More specifically, methods of predicting whether a patient has a specific disease or stage of disease can be improved by determining the quantity of two or more of the following in combination: i) autoantibodies, ii) antigen, iii) or autoantibody-antigen complexes in a sample obtained from a patient. The data collected from the two or more measurements is subjected to statistical analyses wherein the quantity of antigen(s), autoantibody(s) or autoantibody-antigen complexes present in a sample is compared or normalized to a reference set of non-diseased samples enabling the determination of whether a specific disease is present, or alternatively, determining what stage of disease (i.e. disease progression or regression).

In a particular embodiment, the quantities obtained from the measurements are analyzed in multidimensional space (the dimensions of which comprise the responses of the signals from each of the separate assays), and the presence or absence of disease is determined by partitioning the signals on the basis of signal intensity from two or more of the measurements. It is useful to determine appropriate partitioning of data by performing a ROC analysis. A ROC curve is a plot of the true positive rate against the false positive rate for the different possible thresholds of a diagnostic test, wherein the threshold is related to the responses of the signals from said assays. This provides a method of measuring the clinical sensitivity and specificity of a specific subset of data or the data as a whole group. The two or more measurements may consist of measuring variants of autoantibody antigen complexes present in a sample with different capture agents (e.g. antibodies that recognize different epitopes or isoforms of the antigen) and/or different x-human Ig antibodies (e.g. x-IgM versus x-IgG antibodies). The difference between x-IgM and x-IgG antibodies may provide information regarding the stage of disease). Partitioning of signal as described above is also useful when measuring the levels of an antigen and the corresponding antigen-autoantibody complex together. In many cases, the signals may show an inverse relationship since the binding of autoantibodies to an antigen may reduce the amount of antigen which can be detected in a conventional assay.

In an illustrative embodiment, the methods of detecting cancer-associated AAs may be combined with additional diagnostic methods in order to make or confirm a diagnosis of a particular type of cancer, e.g., breast cancer, prostate cancer, ovarian cancer, cervical cancer, skin cancer, pancreatic cancer, colorectal cancer, renal cancer, liver cancer, brain cancer, lymphoma, leukemia, lung cancer, etc. While not wishing to be limited by theory, detection of p53 neopeptides and/or AAs alone would not necessarily differentiate types of cancers. Consequently, detection of p53 neopeptides and/or AAs would provide an means diagnose or confirm a diagnosis of cancer in subjects that may have a positive (or ambiguous) result in screening assays such as PSA, imaging (e.g., mammography), fecal occult blood, or infectious disease (e.g., human papillomavirus).

### Prognostic or Predictive Assays

The disclosure also provides for prognostic (or predictive) assays for determining whether an individual is at risk of developing a condition, disorder or disease associated with the presence or absence of AAs. Such assays can be used for prognostic or predictive purpose, for example to thereby prophylactically treat an individual prior to the onset of a disorder characterized by or associated with AAs, e.g., rheumatoid arthritis, autoimmune disease, and/or cancer. The methods described herein can also be used to determine the levels of such AAs in subjects to aid in predicting the response of such subjects to medication. Another aspect of the invention provides methods for determining an AA expression in an individual to thereby select appropriate therapeutic or prophylactic compounds for that individual.

Accordingly, the prognostic assays described herein can be used to determine whether a subject can be administered a compound (e.g., an agonist, antagonist, peptidomimetic, polypeptide, peptide, nucleic acid, small molecule, or other drug candidate) to treat a disease or condition associated with the presence of AAs. Thus, the invention provides methods for determining whether a subject can be effectively treated with a compound for a disorder or condition associated with aberrant AA levels or in which a test sample is obtained and the AAs are detected using the assays described herein (e.g., wherein the presence, absence, and/or amount of the AAs is diagnostic for a subject that can be administered the compound to treat a disorder associated with an aberrant AA level).

The level of the AAs in a sample obtained from a subject is determined and compared with the level found in a sample obtained from a different subject (or population of subjects) who is free of the condition, in an earlier or later stage of the condition, has a more or less severe form of the condition or responds differently to treatments of the condition. An overabundance (or under abundance) of the AAs in the sample obtained from the subject suspected of having the condition affecting AA levels compared with the sample obtained from the different subject or population is indicative of the condition in the subject being tested.

The methods described herein can be performed, e.g., by utilizing pre-packaged diagnostic kits comprising at least one probe reagent, which can be conveniently used, e.g., in clinical settings for diagnosis or prognosis subjects exhibiting symptoms of the condition.

Correlating a Subject to a Standard Reference Population. To deduce a correlation between clinical response to a treatment and a particular level of AAs, it is necessary to obtain data on the clinical responses exhibited by a population of individuals who received the treatment, i.e., a clinical population. This clinical data may be obtained by retrospective analysis of the results of a clinical trial(s). Alternatively, the clinical data may be obtained by designing and carrying out one or more new clinical trials. The analysis of clinical population data is useful to define a standard reference population(s) which, in turn, are useful to classify subjects for clinical trial enrollment or for selection of therapeutic treatment. In one embodiment, the subjects included in the clinical population have been graded for the existence of the medical condition of interest. Grading of potential subjects can include, e.g., a standard physical exam or one or more lab tests. Alternatively, grading of subjects can include use of a biomarker expression pattern. For example, AA level is a useful as grading criteria where there is a strong correlation between expression pattern and susceptibility or severity to a disease or condition. In one embodiment, a subject is classified or assigned to a particular group or class based on similarity between the measured levels of AA in the subject and the level of the AA observed in a standard reference population.

In one embodiment, a treatment of interest is administered to each subject in a trial population, and each subject's response to the treatment is measured using one or more predetermined criteria. It is contemplated that in many cases, the trial population will exhibit a range of responses, and that the investigator will choose the number of responder groups (e.g., low, medium, high) made up by the various responses. In addition, the expression level of a biomarker (e.g., AAs) is quantified, which may be done before and/or after administering the treatment. These results are then analyzed to determine if any observed variation in clinical response between groups is statistically significant. Statistical analysis methods, which may be used, are described in L. D. Fisher & G. vanBelle, Biostatistics: A Methodology for the Health Sciences (Wiley-Interscience, New York (1993)).

The skilled artisan can construct a mathematical model that predicts clinical response as a function of the level of AAs from the analyses described above. The identification of an association between a clinical response and an expression level for the AAs may be the basis for designing a diagnostic method to determine those individuals who will or will not respond to the treatment, or alternatively, will respond at a lower level and thus may require more treatment, i.e., a greater dose of a drug. The only requirement is that there be a good correlation between the diagnostic test results and the underlying condition. In one embodiment, this diagnostic method uses an assay for AAs described above.

Monitoring Clinical Efficacy. In one embodiment, the present invention provides for monitoring the influence of treatments (e.g., drugs, compounds, small molecules or devices) on the level of AAs. Such assays can also be applied in basic drug screening and in clinical trials. For example, the effectiveness of an agent to increase (or decrease) autoantibody levels can be monitored in clinical trials of subjects. An agent that affects the level of AAs can be identified by administering the agent and observing a response. In this way, the level of the AAs can serve as a marker, indicative of the physiological response of the subject to the agent. Accordingly, this response state may be determined before, and at various points during, treatment of the individual with the agent.

Subject Classification. Standard control levels of AAs are determined by measuring levels in different control groups. The control levels are then compared with the measured level of AAs in a given subject. The subject can be classified or assigned to a particular group based on how similar the measured levels were compared to the control levels for a given group.

As one of skill in the art will understand, there will be a certain degree of uncertainty involved in making this determination. Therefore, the standard deviations of the control group levels can be used to make a probabilistic determination and the method of this invention are applicable over a wide range of probability-based group determinations. Thus, for example, and not by way of limitation, in one embodiment, if the measured level of the AAs falls within 2.5 standard deviations of the mean of any of the control groups, then that individual may be assigned to that group. In another embodiment, if the measured level of the AAs falls within 2.0 standard deviations of the mean of any of the control groups then that individual may be assigned to that group. In still another embodiment, if the measured level of the AAs fall within 1.5 standard deviations of the mean of any of the control groups then that individual may be assigned to that group. In yet another embodiment, if the measured level of the AAs is 1.0 or less standard deviations of the mean of any of the control groups levels then that individual may be assigned to that group. Thus, this process allows determination, with various degrees of probability, which group a specific subject should be placed in, and such assignment would then determine the risk category into which the individual should be placed.

### Substrates

In some embodiments, capture probes may be immobilized on a solid support. Examples of such solid supports include plastics such as polycarbonate, complex carbohydrates such as agarose and sepharose, acrylic resins and such as polyacrylamide and latex beads. Other examples include SurModics Codelink or Schott Hydrogel slides. Alternative solid support materials include magnetic or non-magnetic nano- or micro-particles which are commonly used in homogeneous assays. Techniques for coupling biomolecules to such solid supports are well known in the art (Weir et al., “Handbook of Experimental Immunology” 4th Ed., Blackwell Scientific Publications, Oxford, England, Chapter 10 (1986); Jacoby et al., Meth. Enzym. 34 Academic Press, N.Y. (1974)).

Appropriate linkers, which can be cross-linking agents, for conjugating a ligand to a solid support include a variety of agents that can react with a functional group present on a surface of the support, or with the ligand, or both. Reagents useful as cross-linking agents include homo-bi-functional and, in particular, hetero-bi-functional reagents. Useful bi-functional cross-linking agents include, but are not limited to, N-SLAB, dimaleimide, DTNB, N-SATA, N-SPDP, SMCC and 6-HYNIC. A cross-linking agent can be selected to provide a selectively cleavable bond between a polypeptide and the solid support. For example, a photolabile cross-linker, such as 3-amino-(2-nitrophenyl)propionic acid can be employed as a means for cleaving a polypeptide from a solid support. (Brown et al., Mol. Divers, 4-12 (1995); Rothschild et al., Nucl. Acids Res., 24:351-66 (1996); and U.S. Pat. No. 5,643,722). Other cross-linking reagents are well-known in the art. (See, e.g., Wong (1991), supra; and Hermanson (1996), supra).

A binding agent such as an antibody can be immobilized on a solid support, such as a coated slide, through a covalent amide bond formed between a carboxyl group functionalized substrate and the amino terminus of the polypeptide or, conversely, through a covalent amide bond formed between an amino group functionalized substrate and the carboxyl terminus of the polypeptide. In addition, a bi-functional trityl linker can be attached to the support, e.g., to the 4-nitrophenyl active ester on a resin, such as a Wang resin, through an amino group or a carboxyl group on the resin via an amino resin. Using a bi-functional trityl approach, the solid support can require treatment with a volatile acid, such as formic acid or trifluoracetic acid to ensure that the polypeptide is cleaved and can be removed. In such a case, the polypeptide can be deposited as a patch at the bottom of a well of a solid support or on the flat surface of a solid support.

Hydrophobic trityl linkers can also be exploited as acid-labile linkers by using a volatile acid or an appropriate matrix solution, e.g., a matrix solution containing 3-HPA, to cleave an amino linked trityl group from the polypeptide. Acid lability can also be changed. For example, trityl, monomethoxytrityl, dimethoxytrityl or trimethoxytrityl can be changed to the appropriate p-substituted, or more acid-labile tritylamine derivatives, of the polypeptide, i.e., trityl ether and tritylamine bonds can be made to the polypeptide. Accordingly, a polypeptide can be removed from a hydrophobic linker, e.g., by disrupting the hydrophobic attraction or by cleaving tritylether or tritylamine bonds under acidic conditions, including, if desired, under typical MS conditions, where a matrix, such as 3-HPA acts as an acid.

A binding agent such as an antibody can be conjugated to a solid support through a noncovalent interaction. For example, a magnetic bead made of a ferromagnetic material, which is capable of being magnetized, can be attracted to a magnetic solid support, and can be released from the support by removal of the magnetic field. Alternatively, the solid support can be provided with an ionic or hydrophobic moiety, which can allow the interaction of an ionic or hydrophobic moiety, respectively, with a polypeptide, e.g., a polypeptide containing an attached trityl group or with a second solid support having hydrophobic character.

A solid support can also be provided with a member of a specific binding pair and, therefore, can be conjugated to a polypeptide containing a complementary binding moiety. For example, a bead coated with avidin or with streptavidin can be bound to a polypeptide having a biotin moiety incorporated therein, or to a second solid support coated with biotin or derivative of biotin, such as imino-biotin.

It should be recognized that any of the binding agents disclosed herein or otherwise known in the art can be reversed. Thus, biotin, e.g., can be incorporated into either a polypeptide or a solid support and, conversely, avidin or other biotin binding moiety would be incorporated into the support or the polypeptide, respectively. Other specific binding pairs contemplated for use herein include, but are not limited to, hormones and their receptors, enzyme, and their substrates, a nucleotide sequence and its complementary sequence, an antibody and the antigen to which it interacts specifically, and other such pairs knows to those skilled in the art.

Any suitable substrate may be used and such substrates may be addressable. A plurality of capture probes, each of which can recognize a different target analyte, may be attached to the substrate in an array of spots. If desired, each spot of capture probes may located between two electrodes, the optional label on the detection probe may be a nanoparticle made of a material that is a conductor of electricity, and a change in conductivity may be detected. For example, the electrodes may be made of gold and nanoparticles may be made of gold.

In some embodiments, the methods described herein may detect disease-associated AAs through a specific binding of a nanoparticle-based detection probe with the autoantibody. The signal from the nanoparticles may be amplified with a silver or gold enhancement solution from any substrate which allows observation of the detectable change. Suitable substrates include transparent or opaque solid surfaces (e.g., glass, quartz, plastics and other polymers TLC silica plates, filter paper, glass fiber filters, cellulose nitrate membranes, nylon membranes), and conducting solid surfaces (e.g., indium-tin-oxide (ITO, silicon dioxide (SiO₂), silicon oxide (SiO), silicon nitride, etc.)). The substrate can be any shape or thickness, but generally will be flat and thin like a microscope slide or shaped into well chambers like a microtiter plate. In alternative embodiments, magnetic particles, latex particles, or other types of inorganic or organic particles can be used as a substrate.

### Preparation of Capture Probes

Antibodies that specifically bind to disease-associated antigens, which in turn bind to AAs, can be prepared by methods known to those skilled in the art. Some methods employ polyclonal preparations of antibodies as diagnostic reagents (capture probes), and other methods employ monoclonal isolates. The use of polyclonal mixtures has a number of advantages compared to compositions made of one monoclonal antibody. By binding to multiple sites on an antigen, one can generate a stronger signal (for diagnostics) than a monoclonal that binds to a single site on an antigen. Further, a polyclonal preparation can bind to numerous variants of a prototypical target sequence (e.g., allelic variants, species variants, strain variants, drug-induced escape variants) whereas a monoclonal antibody can bind only to the prototypical sequence or a narrower range of variants thereto. However, monoclonal anti-antigen antibodies are advantageous for detecting a single antigen in the presence or potential presence of closely related antigens.

In methods employing polyclonal antibodies, the preparation typically contains an assortment of binding agents, e.g., antibodies, with different epitope specificities to the target antigen. In some methods employing monoclonal antibodies, it is desirable to have two antibodies of different epitope binding specificities. A difference in epitope binding specificities may be determined by a competition binding assay.

In one embodiment, proteins are isolated from diseased tissues and are used to immunize animals. Consequently, antibodies will be generated to a variety of immunogens from these tissues. The serum from these animals is isolated and deposited onto a solid phase as capture probes. Prior to deposition on the surface, the antibodies can be isolated from the animal sera through techniques well known in the art. If needed, certain populations of antibodies can be isolated through methods such as antigen purification.

To prepare antibodies, a host organism is immunized using the antigen of interest. Antibodies may be raised in any suitable animal (rabbits, goats, mice, chickens, etc.). In suitable embodiments, the antibodies are raised in species that are as evolutionarily removed from humans as possible. The antibodies may also be raised against conserved region of the disease-associated antigen of interest. For instance, conserved regions for the p53 protein were identified in Soussi et al., Oncogene (1990) 5: 945-52.

Adjuvants may be used to enhance effectiveness of the composition include. Typically, the immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect. The proteins may also be incorporated into Immune Stimulating Complexes together with saponins, for example Quil A (ISCOMS).

Immunogenic compositions used to raise antibodies comprise a “sufficient amount” or “an immunologically effective amount” of the antigen of interest, as well as any other of the above mentioned components, as needed. “Immunologically effective amount,” means that the administration of that amount to an individual, either in a single dose or as part of a series, is effective to provoke an immune response and to raise antibodies, as defined above. This amount varies depending upon the health and physical condition of the individual, the taxonomic group of the individual to be treated (e.g., nonhuman primate, primate, rabbit, etc.), the capacity of the organism's immune system to synthesize antibodies, the immunogenicity of the antigenic peptide, and its mode of administration, and other relevant factors. It is expected that the amount will fall in a relatively broad range that can be determined through routine trials. Usually, the amount will vary from 0.01 to 1000 mg/dose, more particularly from 0.1 to 100 mg/dose.

The host serum or plasma is collected following an appropriate time interval to provide a composition comprising antibodies reactive with the peptides of the present invention. The gamma globulin fraction or the IgG antibodies (or Fc domains) can be obtained, for example, by use of saturated ammonium sulfate or DEAE Sephadex, or other techniques known to those skilled in the art.

The monoclonal antibodies can be produced by any hybridoma liable to be formed according to classical methods from spleen cells of an animal, particularly from a mouse or rat, immunized against the peptides of interest, and to be selected by the ability of the hybridoma to produce the monoclonal antibodies recognizing the peptides which has been initially used for the immunization of the animals.

### Detection Probes

In some embodiments, the capture probes bound to the solid support specifically bind to a corresponding molecule to form a complex. Simultaneously or subsequently, the molecule is contacted with a detection probe. In one embodiment, the detection probes are coupled with a label moiety, i.e., detectable group. The particular label or detectable group conjugated to the binding agent is not a critical aspect of the invention, so long as it does not significantly interfere with the specific binding of the binding agent to the target molecule, i.e., human immunoglobulin or disease-associated antigen. In a particular embodiment, the detection probe comprises a nanoparticle conjugated directly or indirectly to an anti-human Ig antibody, e.g., one or more of an anti-IgG (including AAs that possess Fc domains), anti-IgA, anti-IgM, anti-IgE, and anti-IgD. The nanoparticle-antibody conjugate is contacted with the substrate under conditions effective to allow binding of the target molecule (e.g., AAs) on the substrate with the anti-human Ig antibody.

Nanoparticles useful in the practice of the invention include metal (e.g., gold, silver, copper and platinum), semiconductor (e.g., CdSe, CdS, and CdS or CdSe coated with ZnS) and magnetic (e.g., ferromagnetite) colloidal materials. Other nanoparticles useful in the practice of the invention include ZnS, ZnO, TiO₂, AgI, AgBr, HgI₂, PbS, PbSe, ZnTe, CdTe, In₂S₃, In₂, Se₃, Cd₃ P₂, Cd₃ As₂, InAs, and GaAs. The size of the nanoparticles is preferably from about 5 nm to about 150 nm (mean diameter), more preferably from about 5 to about 50 nm, most preferably from about 10 to about 30 nm. The nanoparticles may also be rods. Other nanoparticles useful in the invention include selenium, silica and polymer (e.g., latex) nanoparticles.

Previous studies have demonstrated that biomolecules such as DNA can be conjugated to gold nanoparticles via a thiol linkage (Mirkin et al., Nature 382:607-609 (1996)). The resulting modified gold particles can be used to detect analytes in a variety of formats (See, e.g., Storhoff et al., Chem. Rev., 99:1849-1862 (1999); Niemeyer, C. M. Angew. Chem. Int. Ed., 40:4128-4158 (2001); Liu et al., J. Am. Chem. Soc., 125:6642-6643 (2003)), including DNA microarrays, where high detection sensitivity is achieved in conjunction with silver amplification (Taton et al., Science, 289:1757-1760 (2000); Storhoff et al., Biosens. Bioelectron, 19:875-883 (2004)).

An effective method for functionalizing nanoparticles with biomolecules has been developed. See U.S. Pat. Nos. 6,361,944 and 6,417,340 (Nanosphere, Inc.), which are incorporated by reference in their entirety. The process leads to nanoparticles that are heavily functionalized and have enhanced particle stability. The resulting modified particles have also proven to be very robust as evidenced by their stability in solutions containing elevated electrolyte concentrations, stability towards centrifugation or freezing, and thermal stability when repeatedly heated and cooled. This loading process also is controllable and adaptable. Such methods can also be used to generate nanoparticle-antibody or nanoparticle-biotin conjugates.

In other embodiments, the detectable group can be any material having a detectable physical or chemical property. Such detectable labels have been well-developed in the field of immunoassays and imaging, in general, most any label useful in such methods can be applied to the present invention. Useful labels include magnetic beads (e.g., Dynabeads™), fluorescent dyes (e.g., fluorescein isothiocyanate, Texas red, rhodamine, and the like), radiolabels (e.g., ³H, ¹⁴C, ³⁵S, ¹²⁵I, ¹²¹I, ¹³¹I, ¹¹²In, ^{99m}Tc), other imaging agents such as microbubbles (for ultrasound imaging), ¹⁸F, ¹¹C, ¹⁵O, (for Positron emission tomography), ^{99m}TC, ¹¹¹In (for Single photon emission tomography), enzymes (e.g., horse radish peroxidase, alkaline phosphatase and others commonly used in an ELISA), and calorimetric labels such as colloidal gold or colored glass or plastic (e.g., polystyrene, polypropylene, latex, and the like) beads. Patents that described the use of such labels include U.S. Pat. Nos. 3,817,837; 3,850,752; 3,939,350; 3,996,345; 4,277,437; 4,275,149; and 4,366,241, each incorporated herein by reference in their entirety and for all purposes. See also Handbook of Fluorescent Probes and Research Chemicals (6th Ed., Molecular Probes, Inc., Eugene Oreg.).

The nanoparticle may be linked to an antibody either directly or indirectly. For example, the nanoparticle may be directly functionalized with the desired detection antibody. Alternatively, the nanoparticle may be functionalized with a biotin moiety and the desired detection antibody is also functionalized with a biotin moiety. An avidin or streptavidin molecule is used to link (i.e., “bridge”) the nanoparticle to the antibody. The antibody-nanoparticle conjugate may be formed by step-wise addition of the biotinylated antibody, streptavidin, and biotinylated nanoparticle to the substrate. For example, see U.S. Provisional Application Ser. No. 61/036,892 filed on Mar. 14, 2008, which is hereby incorporated by reference herein in its entirety and U.S. Provisional Application Ser. No. 61/055,875 filed on May 23, 2008, which is hereby incorporated by reference herein in its entirety. Receptor-ligand pairs alternative to streptavidin-biotin also may be used. For instance, the FITC anti-FITC system is a well known alternative to biotin streptavidin. Additionally, double-headed protease inhibitors (Black-eyed pea chymotrypsin or trypsin inhibitor) bind two molecules of protease simultaneously (Gennis et. al., J. Biol. Chem., 251, 741-746). As such, the inhibitors can be used to link the nanoparticle and the antibody using two connecting genetically modified proteases.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds useful as labelling moieties, include, but are not limited to, e.g., fluorescein and its derivatives, rhodamine and its derivatives, dansyl, umbelliferone, and the like. Chemiluminescent compounds useful as labelling moieties, include, but are not limited to, e.g., luciferin, and 2,3-dihydrophthalazinediones, e.g., luminol. For a review of various labeling or signal-producing systems which can be used, see, U.S. Pat. No. 4,391,904.

### Detection and Assays

Means of detecting labels are well known to those of skill in the art. Thus, for example, where the label is a radioactive label, means for detection include a scintillation counter or photographic film for autoradiography. Where the label is a fluorescent label, it can be detected by exciting the fluorochrome with the appropriate wavelength of light and detecting the resulting fluorescence. The fluorescence can be detected visually, by means of photographic film, by the use of electronic detectors such as charge coupled devices (CCDs) or photomultipliers and the like. The fluorescence can be time resolved, or in the case of solution based assays, fluorescence polarization can be used for detection. Furthermore, phosphorescence can be used for detection through the utilization of rare earth elements and there complexes (e.g. europium chelates), upconverting phosphors, or down converting phosphors. Similarly, enzymatic labels can be detected by providing the appropriate substrates for the enzyme and detecting the resulting reaction product. Chemiluminescence detection can be used through labeling of the detection probe with acridinium ester or other chemiluminescent labels known in the art. Finally simple colorimetric labels can be detected simply by observing the color associated with the label.

In some embodiments, assays are homogeneous. Homogeneous assays can involve substrates (e.g. nanoparticles magnetic beads, scintillation proximity assay (SPA) beads), or they may involve the formation of sandwich complexes such as fluorescence resonance energy transfer (FRET) between to fluorophores. A colorimetric method for monitoring scattered light may be used to detect the nanoparticle conjugates. See U.S. Ser. No. 10/995,051, filed Nov. 22, 2004, which is incorporated by reference in its entirety. Moreover, the methods enable the detection of probe-target complexes containing two or more particles in the presence of a significant excess of non-complexed particles, which drives hybridization in the presence of low target concentrations. An alternative homogeneous method detects AAs and their complexes with autoantigens through capture on a magnetic bead surface coated with a binding agent (e.g. antibody). Following capture from the solution of interest (e.g. human serum) on a magnetic bead, the solution can be separated to remove any unbound material. A detection probe, such as antibody coated gold nanoparticle, can be used as a label. Following separation of the complexes, the nanoparticles can be released from the magnetic bead by digesting the proteins in an acid or basic solution, or alternatively, with proteases or other types of enzymes or buffers that would dissociate the antigen/antibody or antibody/antibody complexes. Once released the gold nanoparticles can be detected by monitoring absorbed or scattered light, or through further enlargement via catalytic reduction of a metal (e.g. silver or gold) followed by monitoring the absorbed or scattered light. Alternatively, the nanoparticles are not released from the magnetic bead but detected directly via scattering, flow cytometry, or other methods known in the art. An Alternative homogeneous method may involve labeling of a capture probe with a donor fluorophore, and labeling of a detector probe with an acceptor fluorophore, where the formation of a complex is determined by FRET.

Nanoparticle detection probes, particularly gold nanoparticle probes conjugated to antibodies, are suitable for detection of AAs. A silver-based signal amplification procedure can further provide ultra-high sensitivity enhancement. Silver staining can be employed with any type of nanoparticles that catalyze the reduction of silver and can be used to produce or enhance a detectable change in any assay performed on a substrate, including those described above.

A nanoparticle can also be detected, for example, using resonance light scattering, after illumination by various methods including dark-field microscopy, evanescent waveguides, non-evanescent methods, or planar illumination of glass substrates. Metal particles >40 nm diameter scatter light of a specific color at the surface plasmon resonance frequency (Yguerabide et al., Anal. Biochem., 262:157-176 (1998)), and can be used for multicolor labeling on substrates by controlling particle size, shape, and chemical composition (Taton et al., J. Am. Chem. Soc., 123:5164-5165 (2001); Jin et al., Science, 294:1901-1903 (2001)). In another embodiment, a nanoparticle can be detected in a method of the invention, for example, using surface enhanced raman spectroscopy (SERS) in either a homogeneous solution based on nanoparticle aggregation (Graham et al., Angew. Chem., 112:1103 (2000)), or on substrates in a solid-phase assay (Porter et al., Anal. Chem., 71:4903-4908 (1999)), or using silver development followed by SERS (Mirkin et al., Science, 297:1536-1540 (2002)). In another embodiment, the nanoparticles may be detected by photothermal imaging (Boyer et al., Science, 297:1160-1163 (2002)), diffraction-based sensing technology (Bailey et. al, J. Am Chem. Soc., 125:13541 (2003)), or hyper-Rayleigh scattering (Kim et al., Chem Phys. Lett., 352:421 (2002)).

A nanoparticle can be detected in a method of the invention, for example, using an optical or flatbed scanner. The scanner can be linked to a computer loaded with software capable of calculating grayscale measurements, and the grayscale measurements are calculated to provide a quantitative measure of the amount of analyte detected. Suitable scanners include those used to scan documents into a computer which are capable of operating in the reflective mode (e.g., a flatbed scanner), other devices capable of performing this function or which utilize the same type of optics, any type of grayscale-sensitive measurement device, and standard scanners which have been modified to scan substrates according to the invention. The software can also provide a color number for colored spots and can generate images (e.g., printouts) of the scans, which can be reviewed to provide a qualitative determination of the presence of a nucleic acid, the quantity of a nucleic acid, or both. In addition, it has been found that the sensitivity of assays can be increased by subtracting the color that represents a negative result from the color that represents a positive result.

### Kits

Also within the scope of the disclosure are kits comprising capture and detection probe compositions and instructions for use. The kits are useful for detecting the presence of AAs in a biological sample, e.g., any body fluid including, but not limited to, serum, plasma, lymph, cystic fluid, urine, stool, cerebrospinal fluid, acitic fluid or blood and including biopsy samples of body tissue. For example, the kit can comprise: one or more binding agents specific for disease-associated antigens; means for determining the amount of the AAs in the sample; and means for comparing the amount of the AAs in the sample with a standard. One or more of the detection probes may be labeled. The kit components, (e.g., reagents) can be packaged in a suitable container. The kit can further comprise instructions for using the kit to detect the AAs.

In one embodiment, the kit includes: (1) disease-associated antigen binding agent (e.g., antibody); and (2) an antibody which binds to the AAs and is conjugated (directly or indirectly) to a nanoparticle. The kit can also include, e.g., a buffering agent, a preservative or a protein-stabilizing agent. The kit can further include components necessary for detecting the detectable-label, e.g., an enzyme or a substrate. The kit also can include a calibration set in order to quantitate the amount of autoantibody present in the sample. The calibration set can be programmed into the software as part of the instrumentation. The kit can also contain a control sample or a series of control samples, which can be assayed and compared to the test sample, or to demonstrate that the calibration set is working appropriately. The calibration set may be on the same substrate as the binding agent for the autoantigens, or it may be a reference autoantibody material (or surrogate material that provides a similar response or enables the response to be defined) that is tested and subsequently programmed into the software such that only a control material needs to be provided for a quantitative kit. Each component of the kit can be enclosed within an individual container and all of the various containers can be within a single package, along with instructions for interpreting the results of the assays performed using the kit. The kits may contain a written product on or in the kit container. The written product describes how to use the reagents contained in the kit, e.g., to use the AAs in determining a strategy for preventing or treating a rheumatoid arthritis in a subject. In several embodiments, the use of the reagents can be according to the methods described herein.

### EXAMPLES

The present invention is further illustrated by the following examples, which should not be construed as limiting in any way.

### Example 1

### Preparation of Gold Nanoparticles

Gold colloids (about 15 nm diameter) are prepared by reduction of HAuCl₄ with citrate as described in Grabar, Anal. Chem., 67:735 (1995). Briefly, all glassware is cleaned in aqua regia (3 parts HCl, 1 part HNO₃), rinsed with Nanopure H₂O, then oven dried prior to use. HAuCl₄ and sodium citrate are purchased from Aldrich Chemical Company. Aqueous HAuCl₄ (1 mM, 500 mL) is brought to reflux while stifling. Then, 38.8 mM sodium citrate (50 mL) is added quickly. The solution color changed from pale yellow to burgundy, and refluxing is continued for 15 min. After cooling to room temperature, the red solution is filtered through a Micron Separations Inc. 0.2 micron cellulose acetate filter. Au colloids are characterized by UV-vis spectroscopy using a Hewlett Packard 8452A diode array spectrophotometer and by Transmission Electron Microscopy (TEM) using a Hitachi 8100 transmission electron microscope.

### Example 2

### Preparation of Gold Nanoparticles Coated with Biotins

The following stepwise procedure was used to prepare biotinylated gold nanoparticles:

1. Gold nanoparticles were diluted 1:1 using filtered water and then adjusted to pH 7 by adding 3 μl of 0.2 N NaOH per 1 mL of gold nanoparticle.

2. Two (2) μM of a biotin-PEG-thiol compound (prepared at Nanosphere on an Actapilot Nucleic acid synthesizer using biotin modifiers and PEG phosphoramidites purchased from Glen Research, and a phosphoramidite containing a sulfur linkage prepared at Nanosphere) was added to the 1:1 diluted gold nanoparticles (conc: 6 nM), and the solution was incubated on a bench top shaker at 450 rpm under room temperature conditions.

3. After shaking for 24 hours, a 1.5M NaCl, 100 mM phosphate and 2% Tween20 pH: 7.2 solution was added to the nanoparticles to bring the final salt concentration to 0.15 M NaCl, 10 mM phosphate and 0.2% Tween.

4. After 1 hour of additional incubation, a 1% casein solution was added to the labeled nanoparticles (added 1/10 volume of the total probe solution), and the solution was incubated with shaking for an additional 1 hour at room temperature.

5. After 1 hour, the coated nanoparticles were aliquoted into 1.5 ml low retention tube(s) (0.5 mL per tube) and diluted with 0.01% Tween20 in 1:1 ratio and centrifuged at 12,000 RCF for 25 min at 22° C.

6. The supernatant was removed, and an equal volume of 0.01% Tween20 solution was added to replace the original solution. The centrifugation portion of step 5 was repeated, and the coated nanoparticles were resuspended in assay buffer (1×PBS, 0.1% BSA, 0.3% Tween). The aliquots were combined into a 5 ml glass vial, and the absorbance of the solution at 524 nm was measured using a Nanodrop spectrophotometer. The concentration was calculated according to Beer's law using an extinction coefficient of 2.4×10⁸ M⁻¹ cm⁻¹.

7. After preparation, the probes were stored at 4° C. in assay buffer. Prior to each experiment, a 50 pM probe solution is prepared by diluting the biotinylated probe solution with binding buffer (1×PBS, 1% BSA, 0.3% Tween20)

### Example 3

### Preparation of Codelink Slides Coated with Anti-Filaggrin Antibodies

A rabbit polyclonal anti-filaggrin antibody was purchased from Abcam (Cat # ab24584). The antibodies were deposited onto GE Codelink™ coated glass slides using a GMS417 arrayer (Affymetrix) equipped with a SMP 8xb microspotting pin purchased from Telechem. The antibody was printed at a final concentration of 400 μg/mL by diluting the sample in 1×PBS, 60 mM Trehalose, 0.01% Tween20. Six replicate spots of each antibody were deposited in 16 sub-arrays on GE Codelink™ coated glass slides. The position of the sub-arrays was designed to allow multiple incubation experiments on each slide, achieved by partitioning the slide into separate test wells using Grace proplate slide modules (Cat # 204862). After depositing the antibodies onto the slide surface, the slides were incubated overnight (>12 hours) in a humidity chamber, and then placed in a dessicator at room temperature for storage.

### Example 4

### Assay for the Detection of Anti-Filaggrin Autoantibodies

Experimental. Each slide was assembled into a Grace proplate slide module (Grace Cat. # 204862). All reagent addition steps are performed using a 200 μL multichannel pipette. Unless noted otherwise, reagents were removed from each slide well at the end of each wash or reagent incubation step by inverting and shaking the slide. Binding buffer was 1×PBS, 1% BSA, 0.3% Tween20. Wash buffer was 1×PBS, 0.3% Tween20. All reagent incubations with shaking were performed on a Labnet P4 orbital shaker.

In the first step, the slides with anti-filaggrin antibody were rinsed two times with 200 jut of wash buffer to remove excess antibody. Next, 150 μL of blocking solution (25 mM NaCl/25 mM Tris, pH 8.0/25 mM ethanolamine/0.15% Tween20/0.5×PBS/0.5% BSA) was added to each well of the slide, and the slides were incubated at room temperature (23° C.) while shaking at 250 rpm for 60 min. Each sample was diluted by adding 1 μL of sera to 99 μL of binding buffer. One hundred microliters (100 μL) of each diluted sample is added to a designated well on the slide following removal of the blocking solution, and the slides are incubated at room temperature (23° C.) while shaking at 250 rpm for 60 min. Each slide well is washed twice by adding wash buffer following incubation of the sample. Next, 100 μL of 5 μg/mL Biotinylated x-human IgA1 (Southern biotech, Cat # 9130-08, clone # B3506B4) in binding buffer is added to each well, and the slides are incubated at 23° C. while shaking at 250 rpm for 45 minutes. The slides are washed one time by adding 150 μL of wash buffer following the biotinylated antibody incubation. Next, 100 μL of free streptavidin (SA) (10 ng/μL) in binding buffer is added to each well, and the slides are incubated at 23° C. while shaking at 250 rpm for 10 minutes. The plates are washed two times with wash buffer following SA incubation. Next, 100 μL of a 50 pM Biotin-conjugated gold nanoparticle probe in binding buffer is added to each well, and the slides are incubated at 23° C. with shaking at 250 rpm for 10 min. The nanoparticle solution is removed, and the plates are washed four times with 150 mM NaNO₃, 0.3% Tween20, and two times with 150 mM NaNO₃ adjusted to pH 7.5.

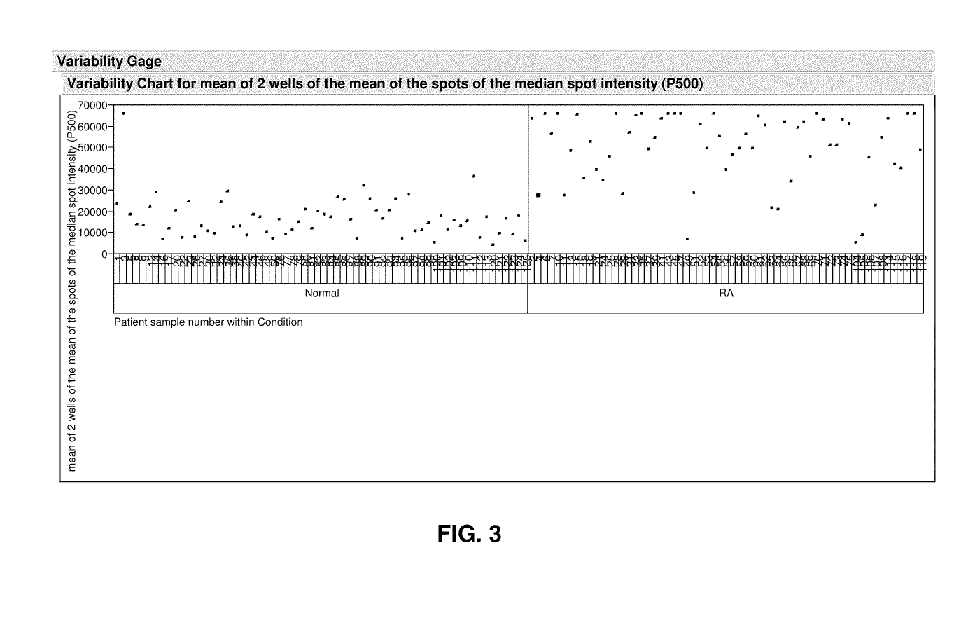

After the final wash, signal enhancement A (Nanosphere Part # E700074D007) and B6 (Nanosphere Part # E700251D001) are mixed in equal volumes, and 150 μL of the mixed reagent is added to each well and incubated for 7 minutes at room temperature. After silver development, the slides are rinsed with copious amounts of deionized water (at least 100 mL/slide). The proplate slide modules are removed, and the slides are dried by spinning in a microfuge. The back of the slides are cleaned with a soft cloth or tissue. Finally, the slides are imaged with the Verigene System using a 20 msec exposure time. Data extraction and quantitation was performed using GenePix software (Axon Instruments). The identity of the samples was blinded to the individual performing the assay to avoid sample bias during the collection of data. For each assay, the median spot intensity was calculated for each of the six replicate antibody spots within a well using Axon genepix analysis software. The mean of the six replicate spots was calculated for each sample. Each sample was assayed in duplicate, and the mean signal intensity of the duplicate sample measurements was plotted for each RA and normal sample, FIG. 3.

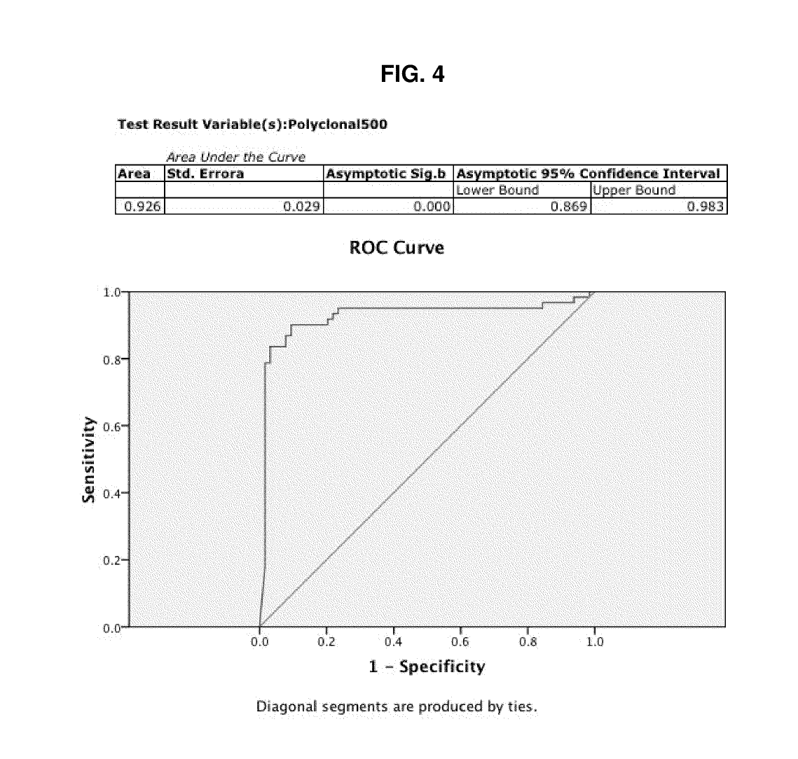

Patient sample testing and results. Using the autoantibody assay described above, we measured autoantibody levels in sera that were collected from patients characterized to have Rheumatoid arthritis (RA). For comparison, we measured autoantibody levels in a control set of sera collected from healthy “normal” patients under the same set of assay conditions. The control and RA samples were purchased from Bioserve and Open Biosystems. Of the 125 samples that were tested in total, 61 of the patients were characterized as having RA. The mean signal intensity for each RA and normal sample is plotted in FIG. 3. A more intense signal is observed in a relatively large proportion of the RA samples when compared to the signal intensity associated with the normal samples. This indicates higher autoantibody levels in the RA patient samples. Based on the signal intensity data from all of the normal and RA samples tested, a receiver operating characteristic (ROC) curve was plotted to model the sensitivity (true positive fraction) versus the false positive fraction (1—specificity) based on these data (FIG. 4). The results show that this assay provides a higher sensitivity and specificity than many of the previously reported methods for diagnosing RA which use either Rheumatoid factor or cyclic citrullinated peptide (CCP) for detection.

### Example 5

### Preparation of Glass Slides Coated with p53 Antigen

p53 antigen was purchased from Santa Cruz biotechnology (sc-4246, full length p53 protein corresponding to amino acids 1-393 of human origin). The antigen was deposited onto GE Codelink™ coated glass slides using a GMS417 arrayer (Affymetrix) equipped with a SMP 8xb microspotting pin purchased from Telechem. The antigen was printed at final concentrations of 30 μg/mL and 1 μg/mL by diluting the sample in 1×PBS, 60 mM Trehalose, 0.01% Tween20. Two replicate spots of each antibody were deposited in 16 sub-arrays on GE Codelink™ coated glass slides. The position of the sub-arrays was designed to allow multiple incubation experiments on each slide, achieved by partitioning the slide into separate test wells using Nanosphere 10 well hybridization gaskets. After depositing the antibodies onto the slide surface, the slides were incubated overnight (>12 hours) in a humidity chamber, and then placed in a dessicator at room temperature for storage.

### Example 6

### Preparation of Glass Slides Coated with x-p53 Antibody

x-p53 antibody 2B2.68 (Santa Crux biotechnology, cat # sc-71817, lot # H1507) was deposited onto glass slides using a GMS417 arrayer (Affymetrix) equipped with a SMP 8xb microspotting pin (Telechem International). The antibody was printed at a final concentration of 1 mg/mL by diluting the sample in 125 mM NaCl, 30 mM phosphate (pH 10), 60 mM Trehalose, 0.001% Tween20. Three replicate spots of each antibody were deposited in 10 sub-arrays on GE Codelink™ coated glass slides. The position of the sub-arrays was designed to allow multiple incubation experiments on each slide, achieved by partitioning the slide into separate test wells using Nanosphere 10 well hybridization gaskets. After depositing the antibodies onto the slide surface, the slides were incubated overnight (>12 hours) in a humidity chamber, and then placed in a dessicator at room temperature for storage.

### Example 7

### Detection of Anti-p53 Autoantibodies and p53 Antigen

Experimental conditions for the detection of anti-p53 autoantibodies (autoantibody test array): Slides prepared as described in Examples 5 and 6 were assembled into a Nanosphere 10 well hybridization gasket. All reagent addition steps were performed using a 200 μL multichannel pipette. Unless noted otherwise, reagents were removed from each slide well at the end of each wash or reagent incubation step by inverting and shaking the slide. Binding buffer is defined as 1×PBS, 1% BSA, 0.3% Tween20. Wash buffer is defined as 1×PBS, 0.3% Tween20. Fifteen (15) nm diameter gold particles coated with streptavidin were purchased from British Biocell International (BBI). All reagent incubations with shaking were performed on a Labnet P4 orbital shaker.

In the first step, 150 μL of blocking solution (50 mM NaCl/50 mM Tris, pH 8.0/50 mM ethanolamine/0.3% Tween20 was added to each well of the slide, and the slides were incubated at room temperature while shaking at 300 rpm for 60 min. The first step was repeated with binding buffer. Next, the slides were spun dry. Samples and calibrators (standards) were prepared as follows: 1) Calibrators were prepared from p53 AAb (purchased as part of a kit from Dianova) and diluted in binding buffer; 2) each sample was diluted by adding 1 μL of sera to 99 μL of binding buffer. The diluted samples and calibrators ere then added to a designated well on the slide, and the slides were incubated at room temperature (23° C.) while shaking at 300 rpm for 20 min. Each slide well was washed twice by adding 150 μL of wash buffer following removal of the sample. Next, 100 μL of a 500 ng/mL solution of Biotinylated x-human goat IgG (Perkin Elmer Life Science, NEF 803) in binding buffer was added to each well, and the slides were incubated at 23° C. while shaking at 300 rpm for 10 minutes. The slides were washed two times by adding 150 μL of wash buffer following the biotinylated antibody incubation. Next, 100 μL of a 50 pM streptavidin-conjugated gold nanoparticle probe solution in binding buffer was added to each well, and the slides were incubated at 23° C. with shaking at 300 rpm for 10 min. The nanoparticle solution was removed and the plates were washed three times with wash buffer, and eight times with 150 mM NaNO₃ adjusted to pH 7.5. After each wash, the solution was removed by inverting the slide and shaking. After the final wash, signal enhancement A (Nanosphere Part # E700074D007) and B6 (Nanosphere Part # E700251D001) were mixed in equal volumes, and 150 μL of the mixed reagent was added to each well and incubated for 6 minutes at room temperature. After signal enhancement, the slides were rinsed with copious amounts of deionized water (at least 100 mL/slide). The proplate slide modules are removed, and the slides were dried by spinning in a microfuge. The back of the slides were cleaned with a soft cloth or tissue.

Experimental conditions for the detection of p53 antigen (p53 antigen test array): Each slide was assembled into a Nanosphere 10 well hybridization gasket. All reagent addition steps were performed using a 200 μL multichannel pipette. Unless noted otherwise, reagents were removed from each slide well at the end of each wash or reagent incubation step by inverting and shaking the slide. Binding buffer was defined as 1×PBS, 1% BSA, 0.3% Tween20. Wash buffer was defined as 1×PBS, 0.3% Tween20. Fifteen (15) nm diameter gold particles coated with streptavidin were purchased from British Biocell International (BBI). All reagent incubations with shaking were performed on a Labnet P4 orbital shaker.

In the first step, the slides with x-p53 antibody were blocked with 200 μL of binding buffer at 35° C. while shaking at 300 rmp for 240 minutes and then spun dry. Next, fifty microliters of each sample (100% serum, no dilution) was added to a designated well on the slide, and the slides are incubated at 35° C. while shaking at 1000 rpm for 120 min. Each slide well was washed three times by adding 150 μL of wash buffer following removal of the sample. Next, 100 μL of 2.5 μg/mL Biotinylated x-p53 antibody was added to each well, and the slides were incubated at 23° C. while shaking at 1000 rpm for 10 minutes. The slides were washed three times by adding 150 μL of wash buffer following the biotinylated antibody incubation. Next, 100 μL of a 50 pM streptavidin-conjugated gold nanoparticle probe solution in binding buffer was added to each well, and the slides were incubated at 23° C. with shaking at 1000 rpm for 10 min. The nanoparticle solution was removed, and the plates were washed three times with wash buffer, and eight times with 150 mM NaNO₃ adjusted to pH 7.5. After each wash, the solution was removed by inverting the slide and shaking. After the final wash, signal enhancement A (Nanosphere Part # E700074D007) and B6 (Nanosphere Part # E700251D001) were mixed in equal volumes, and 150 μL of the mixed reagent was added to each well and incubated for 8 minutes at room temperature. After signal enhancement, the slides were rinsed with copious amounts of deionized water (at least 100 mL/slide). The proplate slide modules were removed, and the slides were dried by spinning in a microfuge. The back of the slides are cleaned with a soft cloth or tissue.

Imaging and Analysis. All slides were imaged with the Verigene System. Data extraction and quantitation was performed using GenePix software (Axon Instruments). For each assay, the median spot intensity was calculated for each of the replicate antibody or antigen spots within a well. The mean signal intensity of the replicate spots within each well was calculated for each sample. The signal intensity for the autoantibody measurements was calculated by subtracting the signal obtained from a 1 μg/mL spotted concentration of p53 from the signal obtained from a 30 μg/mL spotted concentration of p53.

Sample collection. Fifty (50) serum samples from patients diagnosed with prostate or colon cancer were purchased from Bioserve for testing. Fifty (50) samples from patients with no history of cancer were purchased from Bioserve to serve as a normal reference set.

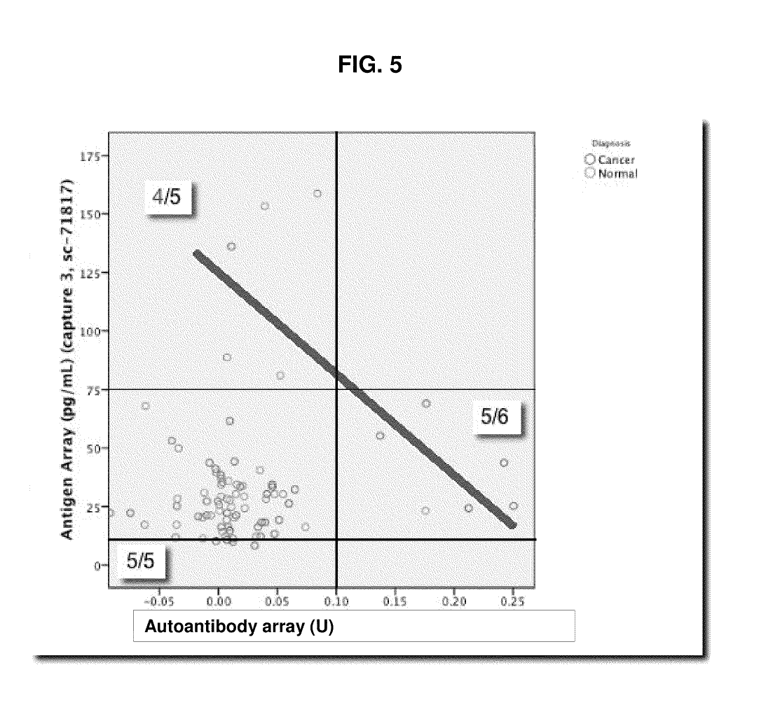

Results. Fifty (50) serum samples that were collected from patients characterized to have cancer were compared to 50 normal patient sera using the two described assays which measured the following: 1) p53 autoantibody levels in serum, and 2) p53 antigen levels in serum. The identity of the samples was blinded to the individual performing the assay to avoid sample bias during the collection of data. The patients were characterized by plotting the signal intensity data from the p53 autoantibody test array (assay 1, x-axis), and the signal intensity from the p53 antigen capture array (assay 2, y-axis) (FIG. 5). The data can be partitioned into six graphical regions based on signal intensity from the two assays. Four (4) samples from normal patients and 1 sample from a cancer patient show a high level of p53 antigen with a low level of p53 auto-antibodies (region 1, left column, top row). By contrast, 5 cancer patients and 1 normal patient show a high level of p53 autoantibodies with a low level of p53 antigen (region 5, right column, middle row). A large proportion of both the cancer and normal patients show a moderate level of antigen and low levels of auto-antibodies.

### Example 8

### Autoantibody Fishing: Assay for the Detection of p53 Antigen-Autoantibody Complexes

Experimental: Each slide was assembled into a Grace proplate slide module (Grace Cat. # 204862). All reagent addition steps were performed using a 200 μL multichannel pipette. Unless noted otherwise, reagents were removed from each slide well at the end of each wash or reagent incubation step by inverting and shaking the slide. Binding buffer is defined as 1×PBS, 1% BSA, 0.3% Tween20. Wash buffer is defined as 1×PBS, 0.3% Tween20. All reagent incubations with shaking were performed on a Labnet P4 orbital shaker.

In the first step, 150 μL of binding buffer was added to each well of the slides with immobilized x-p53 antibodies DO-1 and DO-12, and the slides are incubated at room temperature (23° C.) while shaking at 250 rpm for 60 min. Each sample was diluted by adding 1 μL of sera to 99 uL of binding buffer. One hundred microliters (100 μL) of each diluted sample was added to a designated well on the slide following removal of the blocking solution, and the slides were incubated at room temperature (23° C.) while shaking at 250 rpm for 75 min. Each slide well was washed twice by adding wash buffer following incubation of the sample. Next, 100 μL of a 50 μg/mL mixture of Biotinylated x-human antibodies in binding buffer was added to each well, and the slides were incubated at 23° C. while shaking at 250 rpm for 15 minutes. In one set of experiments, x-human IgG, IgA1, and IgA2 antibodies were added to each well, and in a second set of experiments, a mixture containing x-human IgG and IgM antibodies were added to each well. The slides were washed one time by adding 150 μL of wash buffer following the biotinylated antibody incubation. Next, 100 μL of free streptavidin (SA) (10 ng/μL) in binding buffer is added to each well, and the slides are incubated at 23° C. while shaking at 250 rpm for 5 minutes. The plates were washed two times with wash buffer following SA incubation. Next, 100 μL of a 50 pM Biotin-conjugated gold nanoparticle probe in binding buffer was added to each well, and the slides were incubated at 23° C. with shaking at 250 rpm for 15 min. The nanoparticle solution was removed, and the plates were washed four times with 150 mM NaNO₃, 0.3% Tween20, and two times with 150 mM NaNO₃ adjusted to pH 7.5. After the final wash, signal enhancement A (Nanosphere Part # E700074D007) and B6 (Nanosphere Part # E700251D001) were mixed in equal volumes, and 150 μL of the mixed reagent is added to each well and incubated for 7 minutes at room temperature. Following signal enhancement, the slides were rinsed with copious amounts of deionized water (at least 100 mL/slide). The proplate slide modules were removed, and the slides were dried by spinning in a microfuge. The back of the slides were cleaned with a soft cloth or tissue. Finally, the slides were imaged with the Verigene System. Data extraction and quantitation was performed using GenePix software (Axon Instruments).

The identity of the samples was blinded to the individual performing the assay to avoid sample bias during the collection of data. For each assay, the median spot intensity was calculated for each of the three replicate antibody spots within a well using Axon genepix analysis software. The mean of the three replicate spots was calculated for each sample. Each sample was assayed in duplicate, and the mean signal intensity of the duplicate sample measurements was plotted for each sample.

Patient sample testing and results. We measured the amount of p53 antigen-autoantibody complexes in human sera by capturing p53 antigen from the sample using x-p53 antibodies bound to a glass slide. The autoantibodies bound to p53 were labeled using a mixture of anti-Immunoglobulin antibodies. The bound antigen-antibody complexes were detected using a highly sensitive gold nanoparticle-based detection method (see experimental).

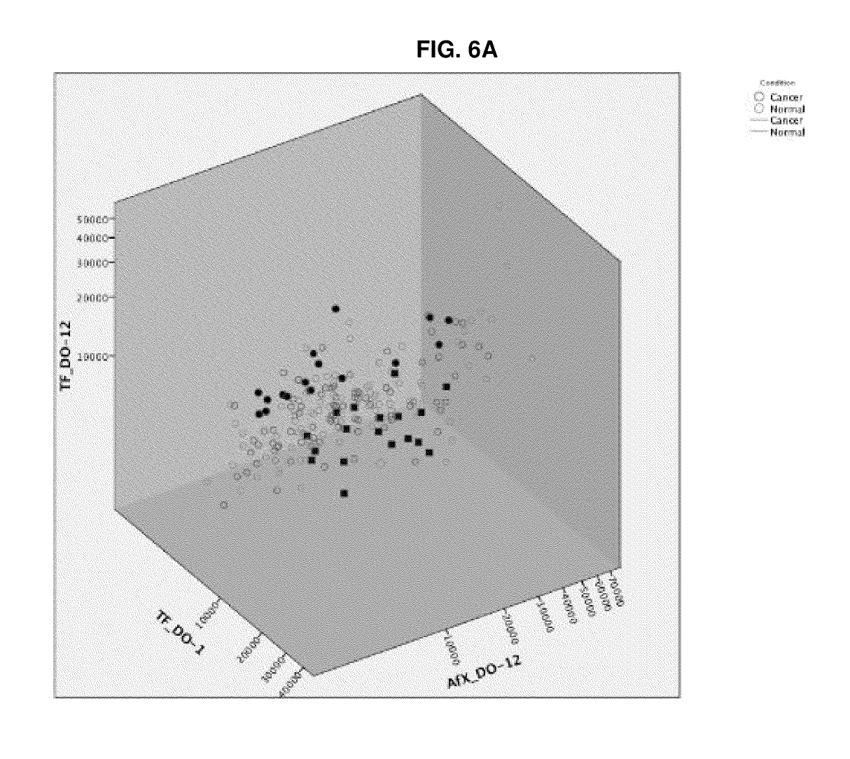

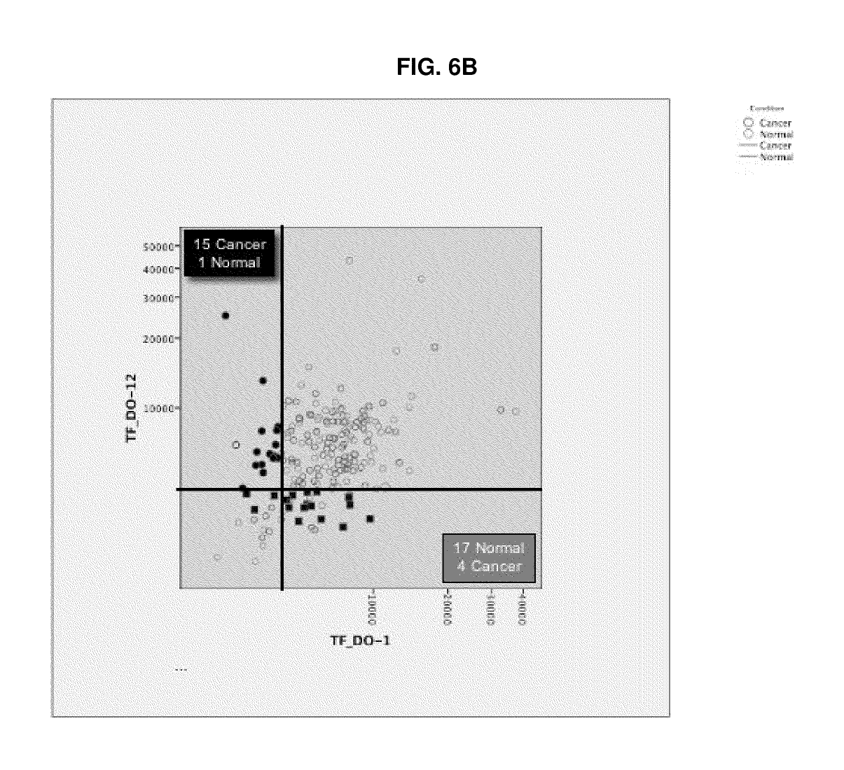

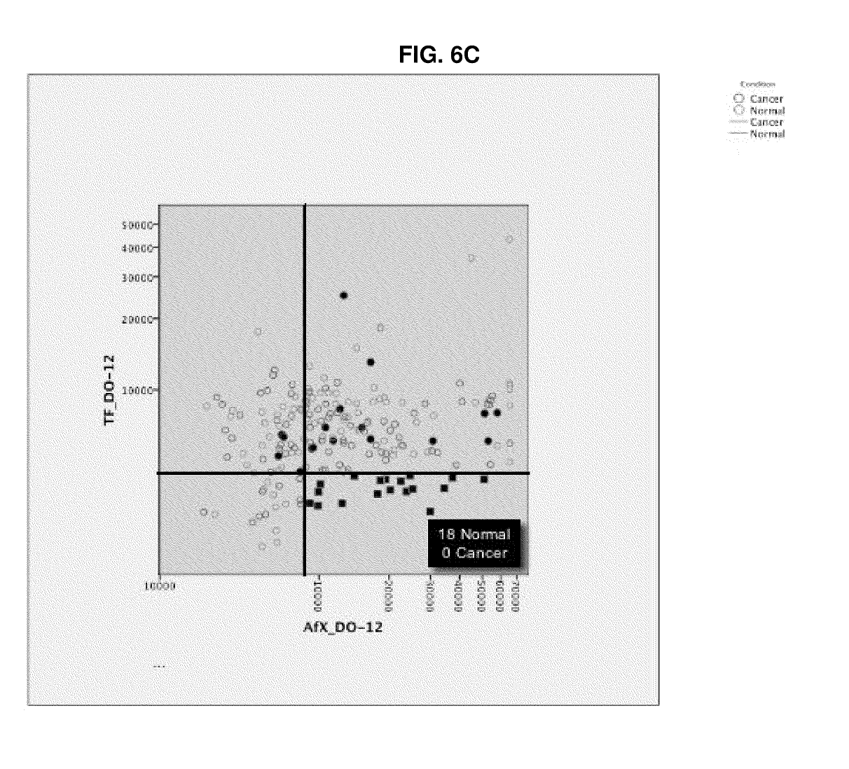

Two antibodies that bind to different epitopes of the p53 antigen(s) were immobilized on the glass slide for comparison. Previous studies have demonstrated that the p53 gene encodes for nine different p53 proteins, and that certain x-p53 antibodies recognize one or more of the specific isoforms of p53 (Bourdon, Brit. J. Of Cancer, 97, 277). The two x-p53 antibodies selected for this experiment (DO-1 and DO-12) recognize different forms of the p53 antigen. As described previously for RA, the concept of binding a conserved region of a specific antigen followed by labeling autoantibodies attached to the antigen is a general strategy for detection of variant forms of the antigen that may not be detectable with conventional sandwich assays which would only recognize wild type forms of the antigen. Additionally, different classes of autoantibodies may be bound to the variant antigen forms since humans produce different classes of immunoglobulins. For instance, IgM antibodies are typically produced early in the course of an infection. Therefore, it may be possible to distinguish diseased from non-diseased states (or stage of disease/infection) by using different immunoglobulins for detection in autoantibody fishing. For this study, we compared signals obtained from two different mixtures of x-human immunoglobins used in the p53 autoantibody fishing assay. We tested 200 sera from patients diagnosed with prostate cancer, colon cancer, or no cancer as a control. FIG. 6A shows the signal intensities obtained from all samples tested using the two x-p53 antibody captures (TF_DO-1 and TF_DO-12) and antibody capture DO-12 labeled with a different mixture of x-human Ig antibodies (labeled Afx_DO-12). Analysis of the 3-dimensional plot shows that certain normal and cancer samples are clustered together based on their response to the different x-p53 capture antibodies and x-human Ig antibodies used in the assay (clusters labeled in black). FIG. 6b shows a two dimensional cross section of the signal from the two different antibodies used to capture p53. By comparing the signal intensity from the two antibody captures, subsets of cancer and normal patients can be distinguished. Additionally, subsets of cancer and normal patients can be distinguished by comparing signal intensities obtained from the same capture antibody (DO-12) with different x-immunoglobulins used for labeling (IgG, IgA mix versus mix containing IgG and IgM), FIG. 6C. These data demonstrate that patients with cancer can be distinguished from normal patients by comparing signals from different sets of capture antibodies and labeling anti-immunoglobulin sets using p53 antigen-autoantibody fishing, in combination with novel partitioning algorithms for distinguishing sets of diseased and non-diseased patients.

### EQUIVALENTS

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can, of course, vary. It is also to be understood that the terminology used herein is for the purpose of describing particular embodiments only, and is not intended to be limiting.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 units refers to groups having 1, 2, or 3 units. Similarly, a group having 1-5 units refers to groups having 1, 2, 3, 4, or 5 units, and so forth.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

All publications, patent applications, patents, and other references mentioned herein are expressly incorporated by reference in their entirety, to the same extent as if each were incorporated by reference individually. In case of conflict, the present specification, including definitions, will control.

What is claimed is:

1. A method for detecting one or more disease-associated autoantibodies present in a sample from a subject comprising:
(a) contacting the sample with (i) a capture probe, wherein the capture probe comprises a first binding agent capable of specifically binding a disease-associated antigen and (ii) a detection probe comprising a second binding agent capable of specifically binding autoantibodies to the disease-associated antigen; and
(b) detecting the presence of a complex formed between the capture probe, the disease-associated antigen, autoantibodies to the disease-associated antigen, and the detection probe, wherein the presence of the complex is indicative of one or more disease-associated autoantibodies in the sample.

2. The method of claim 1, wherein the disease-associated antigen is a polypeptide associated with autoimmune disease.

3. The method of claim 2, wherein the autoimmune disease is selected from the group consisting of: Rheumatoid arthritis, Systemic Lupus erythematosus (SLE), or Grave's disease.

4. The method of claim 2, wherein the polypeptide associated with autoimmune disease is a filaggrin polypeptide, a citrullinated filaggrin polypeptide, or variant thereof.

5. The method of claim 1, wherein the disease-associated antigen is a polypeptide associated with cancer.

6. The method of claim 5, wherein the polypeptide associated with cancer is a p53 polypeptide or variant thereof.

7. The method of claim 1 wherein the first binding agent is an antibody, antibody fragment, aptamer, or polypeptide.

8. The method of claim 7, wherein the first binding agent is a polyclonal antibody raised against the disease-associated antigen.

9. The method of claim 7, wherein the first binding agent is a monoclonal antibody raised against a conserved region of the disease-associated antigen.

10. The method of claim 1, wherein the capture probe is a polyclonal antibody produced in a recombinant system.

11. The method of claim 1, wherein the capture probe is a polyclonal antisera obtained from a mammal immunized with one or more disease-associated marker proteins or variants thereof.

12. The method of claim 11, wherein the disease-associated marker proteins are produced recombinantly.

13. The method of claim 1, wherein the capture probe is a polyclonal antisera obtained from a mammal immunized with diseased tissue obtained from the subject.

14. The method of claim 1, wherein the second binding agent is an anti-human Ig antibody.

15. The method of claim 14, wherein the anti-human Ig antibody is selected from the group consisting of: anti-human IgG, anti-human IgM, anti-human IgA, anti-human IgE, anti-human IgD, or subtypes and mixtures thereof.

16. The method of claim 1 further comprising comparing the levels of the disease-associated autoantibodies in the sample to reference levels of the disease-associated autoantibodies.

17. The method of claim 16, wherein the reference levels are the level of the disease-associated autoantibodies in a control population of subjects unaffected by the disease or medical condition.

18. The method of claim 17, wherein an increase or decrease in the level of the disease-associated autoantibodies compared to the reference level indicates the presence or stage of the disease or medical condition.

19. The method of claim 1, wherein the detection probe further comprises: a nanoparticle conjugated to the second binding agent.

20. The method of claim 19, wherein the nanoparticle is conjugated directly to the second binding agent.

21. The method of claim 19, wherein the nanoparticle is conjugated indirectly to the second binding agent by a bridge or linker molecule.

22. The method of claim 21, wherein the nanoparticle and second binding agent are each conjugated to biotin and the nanoparticle and second binding agent are joined by an avidin or streptavidin bridge.

23. The method of claim 19, wherein the nanoparticle is made of a noble metal.

24. The method of claim 23, wherein the nanoparticle is made of gold or silver.

25. The method of claim 19, wherein the detecting comprises contacting the nanoparticle with silver stain.

26. The method of claim 19, wherein the detecting comprises observing light scattered.

27. The method of claim 1, wherein the detection probe further comprises a fluorophore, a phosphor, a quantum dot, or an enzyme conjugate.

28. The method of claim 1, wherein the sample is blood, plasma, or serum

29. The method of claim 1, wherein the subject is a human.

30. The method of claim 1, wherein the capture probe is bound to a substrate.

31. The method of claim 30, wherein the substrate is a nanoparticle, a thin film, or a magnetic bead.

32. The method of claim 30, wherein the substrate has a planar surface.

33. The method of claim 30, wherein the substrate is made of glass, quartz, ceramic, or plastic.

34. The method of claim 30, wherein the substrate is addressable.

35. The method of claim 1, wherein the sample is first contacted with the detection probe and then contacted with the capture probe.

36. The method of claim 1, wherein the sample is first contacted with the capture probe and then contacted with the detection probe.

37. The method of claim 1, wherein the sample, the detection probe, and the capture probe are contacted simultaneously.

38. The method of claim 1, wherein the complex is detected by photonic, electronic, acoustic, opto-acoustic, gravitic, electro-chemical, electro-optic, mass-spectrometric, magnetic, paramagnetic, enzymatic, chemical, biochemical, or physical means.

39. A method for diagnosing or monitoring a disease or medical condition associated with autoantibodies in a subject, the method comprising:
(a) measuring the level of one or more disease-associated antigens in a sample from the subject;
(b) measuring the level of one or more disease-associated autoantibodies in the sample; and
(c) comparing the levels of the disease-associated antigens and disease-associated autoantibodies in the sample to reference levels of the disease-associated antigens and disease-associated autoantibodies, wherein the presence, absence, or stage of a disease or medical condition is indicated by a difference between the reference levels and the levels of the disease-associated antigens and disease-associated autoantibodies in the sample.

40. The method of claim 39,
wherein measuring the level of the one or more disease-associated antigens is by contacting the sample with (i) a first capture probe bound to a substrate, wherein the first capture probe comprises a first binding agent capable of specifically binding to the disease-associated antigen and (ii) a first detection probe comprising a second binding agent capable of specifically binding to the disease-associated antigen; and
wherein measuring the level of the one or more disease-associated autoantibodies is by contacting the sample with (i) a second capture probe bound to a substrate, wherein the second capture probe comprises a third binding agent capable of specifically binding to the disease-associated autoantibodies and (ii) a second detection probe comprising a fourth binding agent capable of specifically binding to the disease-associated autoantibodies.

41. The method of claim 40, wherein the first binding agent is an antibody raised against the disease-associated antigen.

42. The method of claim 41, wherein the second binding agent is an antibody raised against the disease-associated antigen, and wherein the first binding agent and the second binding agent may be the same or different.

43. The method of claim 40, wherein the third binding agent is the disease-associated antigen, and the fourth binding agent is an anti-human Ig antibody.

44. The method of claim 43, wherein the anti-human Ig antibody is selected from the group consisting of: anti-human IgG, anti-human IgM, anti-human IgA, anti-human IgE, anti-human IgD, and subtypes and mixtures thereof.

45. The method of claim 39, wherein the reference levels are the level of the disease-associated autoantibodies and the level of the disease-associated antigens in a control population of subjects unaffected by the disease or medical condition.

46. The method of claim 45, wherein (i) an increase or decrease in the level of the disease-associated antigens compared to the reference level and (ii) an increase or decrease in the level of the disease-associated autoantibodies compared to the reference level indicates the presence, absence, or stage of the disease or medical condition.

47. The method of claim 40, wherein the first binding agent is p53, the second binding agent is a x-p53 antibody, the third binding agent is a x-p53 antibody, and the fourth binding agent is an anti-human Ig antibody.

48. The method of claim 47, wherein (i) an increase or decrease between the level of p53 antigen compared to the reference level and (ii) an increase or decrease in the level of p53 autoantibodies compared to the reference level indicates the presence or stage of cancer.

49. The method of claim 48, wherein the cancer is selected from the group consisting of:
prostate, breast, colon, cervical, and lung cancer.

50. A method for predicting whether a subject has a specific disease or to determine the stage of disease, comprising the steps of:
(a) measuring the level of at least two biomarkers selected from the group consisting of: (i) one or more disease-associated autoantibodies, (ii) one or more disease-associated antigens, and (iii) one or more autoantibody-antigen complexes in a sample obtained from the subject;
(b) analyzing in levels of the biomarkers from the sample and the levels of the biomarkers in one or more reference standards in multidimensional space, wherein each dimension of the multidimensional space corresponds to the level of a single biomarker; and
(c) partitioning the plotted levels of the biomarkers from the sample and the one or more reference standards to determine whether the subject has a specific disease or to determine the stage of disease.

51. The method of claim 50, wherein the partitioning is by performing a receiver operating characteristic (ROC) analysis.

52. The method of claim 50, wherein the partitioning is by CART, CRT, or CHAID analysis.

53. The method of claim 50, wherein the measuring the level of at least two biomarkers comprises measuring the level of autoantibody-antigen complexes with multiple capture probes or detection probes.

54. The method of claim 53, wherein the multiple capture probes include two different antibodies that bind to separate epitopes of the same antigen.

55. The method of claim 53, wherein the multiple detection probes include different anti-human Ig antibodies or mixtures thereof.
